(19) United States
(12) Patent Application Publication
(10) Pub. No.: US 2025/0194964 A1
(43) Pub. Date: Jun. 19, 2025

(54) GLUCOSE OXIDASE SENSORS WITH IMPROVED STABILITY

(71) Applicant: Abbott Diabetes Care, Inc., Alameda, CA (US)

(72) Inventors: Phu Le, Alameda, CA (US); John LaTour, Alameda, CA (US); Soya Gamsey, Alameda, CA (US); Mark Yahnke, Alameda, CA (US); Albert Gutes-Regidor, Alameda, CA (US); Balasubrahmanya S. Bommakanti, Alameda, CA (US); Emmanuel Perez, Alameda, CA (US); Udo Hoss, Alameda, CA (US)

(21) Appl. No.: 18/987,112

(22) Filed: Dec. 19, 2024

Related U.S. Application Data
(60) Provisional application No. 63/611,883, filed on Dec. 19, 2023.

(51) Int. Cl. A61B 5/145; A61B 5/1486

(52) U.S. Cl. CPC A61B 5/14546; A61B 5/14532; A61B 5/1486

(57) ABSTRACT

The present invention relates generally to systems, devices, and methods for in vivo monitoring of an analyte level. In particular, the present invention relates to sensors having saccharides or similar molecules present in glucose-oxidase containing sensing layers to improve sensor stability.

### CROSS-REFERENCE TO RELATED APPLICATIONS

The present application claims the priority benefit of U.S. Provisional Patent Application No. 63/611,883, filed Dec. 19, 2023, which is incorporated by reference in its entirety.

### FIELD

The present invention relates generally to systems, devices, and methods for in vivo monitoring of an analyte level. In particular, the present invention relates to sensors having saccharides or similar molecules present in glucose-oxidase containing sensing layers to improve sensor stability and performance.

### BACKGROUND

The detection of various analytes within an individual can sometimes be vital for monitoring the condition of their health. Deviation from normal analyte levels can often be indicative of a number of physiological conditions. Glucose levels, for example, can be particularly important to detect and monitor in diabetic individuals. By monitoring glucose levels with sufficient regularity, a diabetic individual may be able to take corrective action (e.g. by injecting insulin to lower glucose levels or by eating to raise glucose levels) before significant physiological harm occurs. Monitoring of other analytes may be desirable for other various physiological conditions. Monitoring of multiple analytes may also be desirable in some instances, particularly for comorbid conditions resulting in simultaneous dysregulation of two or more analytes in combination with one another.

Many analytes represent intriguing targets for physiological analyses, provided that a suitable detection chemistry can be identified. To this end, in vivo analyte sensors configured for assaying various physiological analytes have been developed and refined over recent years, many of which utilize enzyme-based detection strategies to facilitate detection specificity. Indeed, in vivo analyte sensors utilizing a glucose-responsive enzyme for monitoring blood glucose levels are now in common use among diabetic individuals. In vivo analyte sensors for other analytes are in various stages of development, including in vivo analyte sensors capable of monitoring multiple analytes. However, substances in biological fluid can interfere with enzyme-based detection of the analyte, such as glucose. Moreover, sensor stability and lifetime are limited, such as by degradation or impaired performance of the enzyme or other components of the sensor over time. Accordingly, improved in vivo analyte sensors with improved stability and performance are needed.

### SUMMARY

In some aspects, provided herein are analyte sensors comprising a sensing layer and a membrane polymer, wherein the sensing layer comprises glucose oxidase enzyme and wherein the sensing layer and/or the membrane polymer comprise a carbohydrate. Such analyte sensors are referred to herein as “doped” with the carbohydrate. For example, an analyte sensor having a sensing layer and/or membrane polymer comprising lactose is referred to as a “lactose-doped” sensor. As another example, an analyte sensor having a sensing layer and/or membrane polymer comprising glucose is referred to as a “glucose-doped” sensor.

Any suitable carbohydrate may be used. In some embodiments, the carbohydrate is a pyranose, furanose, disaccharide, oligosaccharide, polysaccharide, iminosugar, sugar alcohol, glycoside, amino sugar, or a combination thereof. In some embodiments, the pyranose is glucose, galactose, mannose, or a combination thereof. In some embodiments, the furanose is fructose, xylose, ribose, or a combination thereof. In some embodiments, the disaccharide is lactose, maltose, cellobiose, or a combination thereof. In some embodiments, the oligo- or polysaccharide is starch, maltotriose, amylose, cellulose, inulin, or a combination thereof. In some embodiments, the iminosugar is nojirimycin. In some embodiments, the sugar alcohol is inositol. In some embodiments, the glycoside is a glucoside. For example, in some embodiments the glucoside is 1-O-methyl glucose. In some embodiments, the glycoside comprises an aryl group. In some embodiments, the the glycoside comprises an acyl group. In some embodiments, the amino sugar is a glucosamine or N-acetylglucosamine.

In some embodiments, the sensing layer comprises a redox polymer. In some embodiments, the redox polymer comprises a transition metal complex, a polymeric backbone, and a crosslinker. In some embodiments, the transition metal complex is an osmium complex. In some embodiments, the transition metal complex has the following formula:

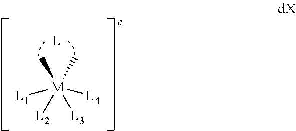

wherein
(i) M is osmium; and
(ii) L is selected from the group consisting of:

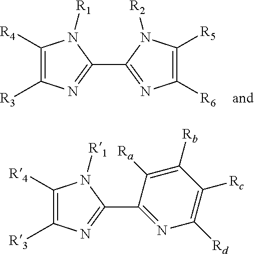

wherein:
R₁, R₂, and R′₁ are independently substituted or unsubstituted alkyl, alkenyl, or aryl groups;
R₃, R₄, R₅, R₆, R′₃, R′₄, Rₐ, R_{b}, R_{c}, and R_{d} are independently —H, —F, —Cl, —Br, —I, —NO₂, —CN, —CO₂H, —SO₃H, —NHNH₂, —SH, aryl, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl;
c is an integer selected from −1 to −5 or +1 to +5 indicating a positive or negative charge;
X represents at least one counter ion;
d is an integer from 1 to 5 representing the number of counter ions; and
L₁, L₂, L₃ and L₄ are ligands,
wherein L₁ comprises a heterocyclic compound coupled to the polymeric backbone; and
wherein L₁ and L₂ in combination form a first bidentate ligand.
   In some embodiments, the M is osmium and the transition complex has the following formula:

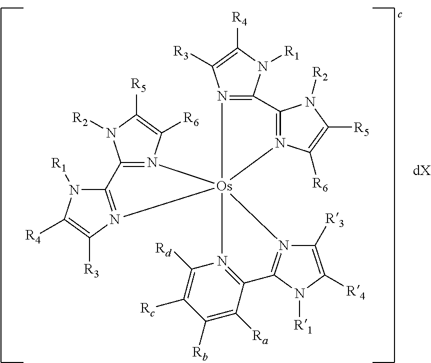

wherein R₃, R₄, R₅, R₆, Rₐ, R_{b}, R_{c}, R_{d}, R′₃ and R′₄ are H;
R₁ and R₂ are independently substituted or unsubstituted C1 to C12 alkyls; and
R′₁ is independently —H or substituted or unsubstituted C1-C12 alkoxy, C1-12 alkylthio, C1-C12 alkylamino, C2-C24 dialkylamino, or C1-C12 alkyl.

In some embodiments, at least one of R₁, R₂, and R′₁ comprises a reactive group selected from the group consisting of carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxyamine, alkyl halide, imidazole, pyridine, phenol, alkyl sulfonate, halotriazine, imido ester, maleimide, hydrazide, hydroxy, and photo-reactive azido aryl groups. In some embodiments, the at least one of R₁, R₂, and R′₁ is coupled to the polymeric backbone.

In some embodiments, the crosslinker is an epoxide. In some embodiments, the crosslinker is polyethylene glycol diglycidylether (PEGDGE), glycerol triglycidyl ether (Gly3).

In some embodiments, the polymeric backbone is a poly(vinylpyridine). In some embodiments, the the polymeric backbone is a poly(vinylpyridine) having the structure:

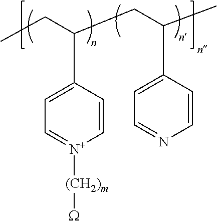

wherein m is 1 to 18, n and n′ are the average number of pyridinium and pyridine
subunits and n″ is the number of repeating polymer units.

In some embodiments, the redox polymer has the following structure:

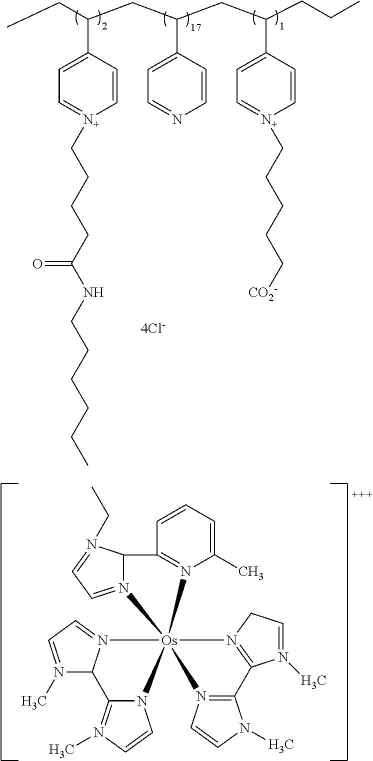

In some embodiments, the membrane polymer comprises the following structure:

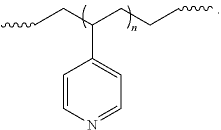

In some embodiments, the membrane polymer comprises a poly(vinylpyridine-co-styrene) copolymer. In some embodiments, the membrane polymer is derivatized by the addition of propylsulfonate and poly(ethyleneoxide) moieties.

In some embodiments, the membrane polymer comprises the following structure:

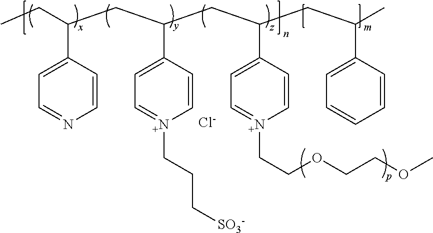

wherein x is 0.85, y is 0.1, z is 0.05, n is 9, m is 1, and p is about 10.

In some aspects, provided herein is a method of manufacturing an analyte sensor described herein. In some embodiments, provided herein is a method of manufacturing an analyte sensor comprising contacting a sensing layer of the analyte sensor with a carbohydrate, wherein the sensing layer comprises glucose oxidase enzyme. In some embodiments, contacting comprises contacting the sensing layer with a solution comprising the carbohydrate, followed by coating the sensing layer with a membrane polymer. In some embodiments, coating the sensing layer with a membrane polymer comprises dipping the sensing layer in a solution comprising a membrane polymer and a crosslinker.

In some embodiments, contacting comprises contacting the sensing layer with a solution comprising the carbohydrate, a membrane polymer, and a crosslinker. In some embodiments, the crosslinker is polyethylene glycol diglycidylether (PEGDGE), glycerol triglycidyl ether (Gly3). In some embodiments, the carbohydrate diffuses from the solution into the sensing layer.

In some embodiments, the analyte sensor comprises a sensing layer coated with a membrane polymer, contacting comprises contacting the electrochemical sensor device with a solution comprising the carbohydrate, wherein the carbohydrate diffuses through the membrane polymer and into the sensing layer.

In some embodiments, the membrane polymer comprises the following structure:

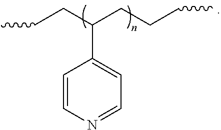

In some embodiments, the membrane polymer comprises a poly(vinylpyridine-co-styrene) copolymer. In some embodiments, the membrane polymer is derivatized by the addition of propylsulfonate and poly(ethyleneoxide) moieties.

In some embodiments, the membrane polymer comprises the following structure:

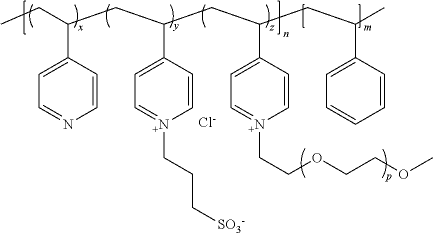

wherein x is 0.85, y is 0.1, z is 0.05, n is 9, m is 1, and p is about 10.

In some embodiments, the sensing layer comprises a redox polymer. In some embodiments, the redox polymer comprises a transition metal complex, a polymeric backbone, and a crosslinker. In some embodiments, the transition metal complex is an osmium complex.

In some embodiments, the transition metal complex has the following formula:

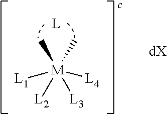

wherein
(i) M is osmium; and
(ii) L is selected from the group consisting of:

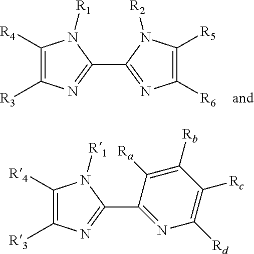

wherein:
R₁, R₂, and R′₁ are independently substituted or unsubstituted alkyl, alkenyl, or aryl groups;
R₃, R₄, R₅, R₆, R′₃, R′₄, Rₐ, R_{b}, R_{c}, and R_{d} are independently —H, —F, —Cl, —Br, —I, —NO₂, —CN, —CO₂H, —SO₃H, —NHNH₂, —SH, aryl, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl;
c is an integer selected from −1 to −5 or +1 to +5 indicating a positive or negative charge;
X represents at least one counter ion;
d is an integer from 1 to 5 representing the number of counter ions; and
L₁, L₂, L₃ and L₄ are ligands,
wherein L₁ comprises a heterocyclic compound coupled to the polymeric backbone; and
wherein L₁ and L₂ in combination form a first bidentate ligand.

In some embodiments, M is osmium and the transition complex has the following formula:

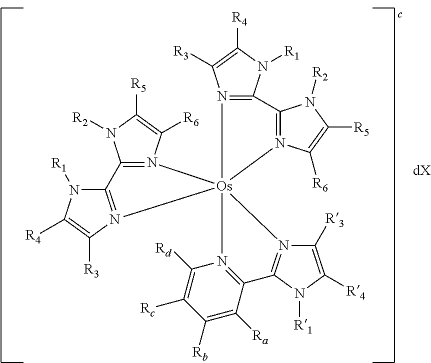

wherein R₃, R₄, R₅, R₆, Rₐ, R_{b}, R_{c}, R_{d}, R′₃ and R′₄ are H;
R₁ and R₂ are independently substituted or unsubstituted C1 to C12 alkyls; and
R′₁ is independently H or substituted or unsubstituted C1-C12 alkoxy, C1-12 alkylthio, C1-C12 alkylamino, C2-C24 dialkylamino, or C1-C12 alkyl.

In some embodiments, at least one of R₁, R₂, and R′₁ comprises a reactive group selected from the group consisting of carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxyamine, alkyl halide, imidazole, pyridine, phenol, alkyl sulfonate, halotriazine, imido ester, maleimide, hydrazide, hydroxy, and photo-reactive azido aryl groups. In some embodiments, at least one of R₁, R₂, and R′₁ is coupled to the polymeric backbone.

In some embodiments, the crosslinker is an epoxide. In some embodiments, the crosslinker is polyethylene glycol diglycidylether (PEGDGE), glycerol triglycidyl ether (Gly3).

In some embodiments, the polymeric backbone is a poly(vinylpyridine). In some embodiments, the polymeric backbone is a poly(vinylpyridine) having the structure:

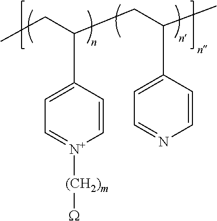

wherein m is 1 to 18, n and n′ are the average number of pyridinium and pyridine
subunits and n″ is the number of repeating polymer units.

In some embodiments, the redox polymer has the following structure:

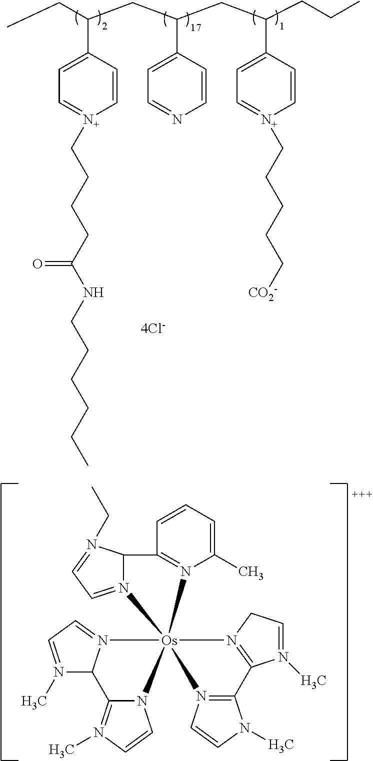

The analyte sensors provided herein may be provided in a package. For example, the analyte sensor may contained in a medical device.

The analyte sensors provided herein find use in a method of monitoring blood glucose levels in a subject. For example, in some embodiments provided herein are methods of monitoring blood glucose levels in a subject, comprising contacting the subject with an analyte sensor provided herein.

### DESCRIPTION OF THE DRAWINGS

FIG. 1 shows a diagram of an illustrative sensing system that may incorporate an analyte sensor of the present disclosure.

FIGS. 2A-2C show cross-sectional diagrams of analyte sensors comprising a single active area.

FIGS. 3A-3C show cross-sectional diagrams of analyte sensors comprising two active areas.

FIG. 4 shows a cross-sectional diagram of an analyte sensor comprising two working electrodes, each having an active area present thereon

FIG. 5 is a diagram showing a top view of an exemplary carbon working electrode having an active area thereon.

FIG. 6A shows drift stability and FIG. 6B shows shelf life stability for sensors containing lactose in the sensing layer (e.g. lactose doped sensors) vs. commercially available glucose sensors.

FIG. 7A shows drift stability and FIG. 7B shows shelf life stability for sensors containing glucose, fructose, or sucrose in the sensing layer vs. commercially available glucose sensors.

FIG. 8 shows sensor stability and shelf life comparing various carbohydrates added to either the sensing layer or the polymeric membrane of a sensor device.

### DETAILED DESCRIPTION

### 1. Definitions

Unless otherwise indicated, all numbers expressing quantities and the like in the present specification and associated claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e. each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, tools and methods are well adapted to attain the ends and advantages mentioned as well as those that are inherent therein. The particular embodiments disclosed herein are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, tools and methods illustratively disclosed herein may suitably be practiced in the absence of any element that is not specifically disclosed herein and/or any optional element disclosed herein.

While systems, tools and methods are described in terms of “comprising,” “containing,” or “including” various components or steps, the systems, tools and methods can also “consist essentially of” or “consist of” the various components and steps.

All numbers and ranges disclosed herein may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values.

The terms in the claims have their plain, ordinary meaning unless otherwise explicitly and clearly defined by the patentee. The indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

### 2. Analyte Sensors

In some aspects, provided herein are analyte sensors with improved stability and performance. In some embodiments, provided herein are analyte sensors comprising a sensing layer and a membrane polymer. The terms “analyte sensor” and “biosensor” are used interchangeably herein.

The terms “sensing layer”, “active area”, and “analyte-responsive active area” are used interchangeably herein and refer to the area of the sensor comprising an enzyme or enzyme system configured for assaying one or more analytes of interest. Accordingly, the sensing layer (i.e. active area) refers to the portion of the sensor where interaction of the analyte with the enzyme or enzyme system occurs. In some embodiments, the sensing layer comprises glucose oxidase enzyme. In some embodiments, the sensing layer comprises the enzyme or enzyme system for assaying one or more analytes of interest (e.g. glucose oxidase enzyme) and a redox polymer. Exemplary redox polymers are described in more detail in a separate section below.

The terms “membrane polymer” and “polymeric membrane” are used interchangeably herein and refer to a polymer material disposed over the sensing layer. Accordingly, in some embodiments, the membrane polymer coats the sensing layer (e.g. the active area). In some embodiments, during use of the device the sample fluid first comes into contact with the membrane polymer, and subsequently passes through the membrane polymer and onto the sensing layer. In some embodiments, the membrane polymer limits the flux of analytes onto the sensing layer. Such a membrane polymer that limits the flux of analytes onto the sensing layer may be referred to as a “mass transport limiting membrane”, an “analyte-restricting membrane”, an “analyte-flux limiting membrane”, and the like. The performance of a biosensor described herein can be complicated at high rates of analyte flux. For example, at high rates of glucose flux, an amperometric glucose biosensor may be kinetically overwhelmed, such that the relationship between the concentration of glucose in a sample fluid and the response from the biosensor becomes non-linear. This kinetic problem may be solved by the interposition of an analyte-flux-limiting membrane between the sample fluid and the sensing layer of the biosensor.

In some embodiments, the sensing layer and/or the membrane polymer comprise a carbohydrate. In some embodiments, the sensing layer comprises a carbohydrate. In some embodiments, the membrane polymer comprises a carbohydrate, and the carbohydrate diffuses from the membrane polymer into the sensing layer prior to or during use of the device. In some embodiments, the analyte sensor comprising a carbohydrate displays increased stability and performance compared to an equivalent analyte sensor lacking the carbohydrate in the sensing layer and/or membrane polymer.

Before describing the analyte sensors of the present disclosure in further detail, a brief overview of exemplary in vivo analyte sensor configurations and sensor systems employing the analyte sensors will be provided first so that the embodiments of the present disclosure may be better understood.

### Exemplary Analyte Sensor Configurations

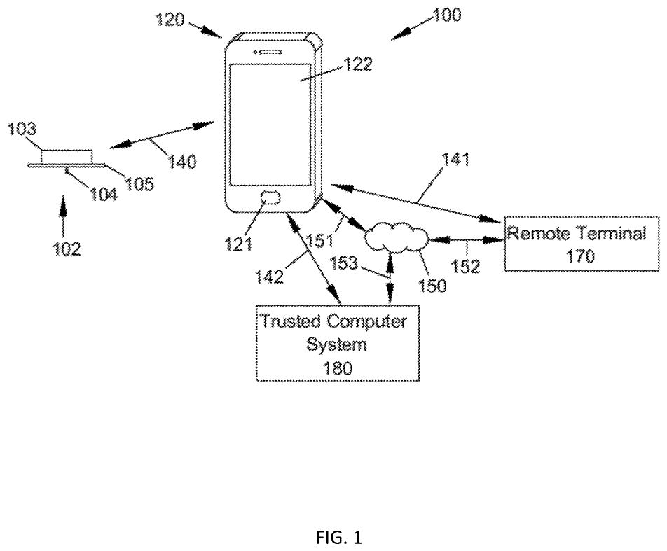

FIG. 1 shows a diagram of an illustrative sensing system that may incorporate an analyte sensor of the present disclosure. As shown, sensing system 100 includes sensor control device 102 and reader device 120 that are configured to communicate with one another over local communication path or link 140, which may be wired or wireless, uni- or bi-directional, and encrypted or non-encrypted. Reader device 120 may constitute an output medium for viewing analyte concentrations and alerts or notifications determined by sensor 104 or a processor associated therewith, as well as allowing for one or more user inputs, according to some examples. Reader device 120 may be a multi-purpose smartphone or a dedicated electronic reader instrument. While only one reader device 120 is shown, multiple reader devices 120 may be present in certain examples. Reader device 120 may also be in communication with remote terminal 170 and/or trusted computer system 180 via communication path(s)/link(s) 141 and/or 142, respectively, which also may be wired or wireless, uni- or bi-directional, and encrypted or non-encrypted. Reader device 120 may also or alternately be in communication with network 150 (e.g. a mobile telephone network, the internet, or a cloud server) via communication path/link 151. Network 150 may be further communicatively coupled to remote terminal 170 via communication path/link 152 and/or trusted computer system 180 via communication path/link 153. Alternately, sensor 104 may communicate directly with remote terminal 170 and/or trusted computer system 180 without an intervening reader device 120 being present. For example, sensor 104 may communicate with remote terminal 170 and/or trusted computer system 180 through a direct communication link to network 150, according to some examples, as described in U.S. Patent Application Publication 2011/0213225 and incorporated herein by reference in its entirety. Any suitable electronic communication protocol may be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 may be accessible, according to some examples, by individuals other than a primary user who have an interest in the user's analyte levels. Reader device 120 may comprise display 122 and optional input component 121. Display 122 may comprise a touch-screen interface, according to some examples.

Sensor control device 102 includes sensor housing 103, which may house circuitry and a power source for operating sensor 104. Optionally, the power source and/or active circuitry may be omitted. A processor (not shown) may be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some examples.

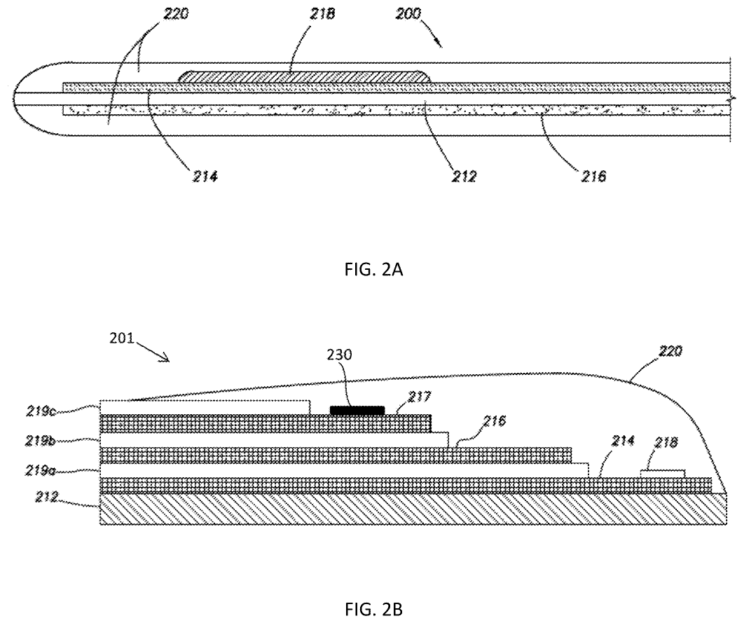

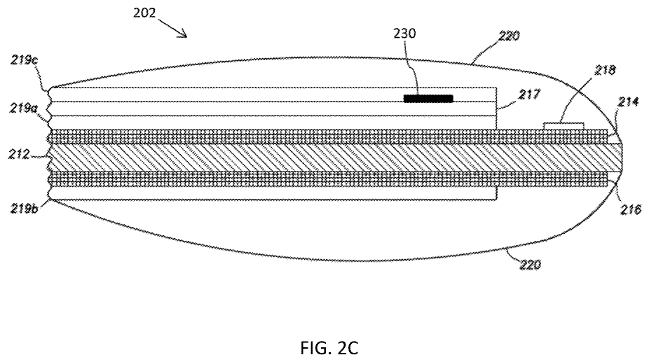

In some embodiments, sensor 104 is adapted to be at least partially inserted into a tissue of interest, such as within the dermal or subcutaneous layer of the skin. Alternately, sensor 104 may be adapted to penetrate the epidermis. Still further alternately, sensor 104 may be disposed superficially and not penetrate a tissue, such as when assaying one or more analytes in perspiration upon the skin. Sensor 104 may comprise a sensor tail of sufficient length for insertion to a desired depth in a given tissue. The sensor tail may comprise at least one working electrode and an active area (e.g. an analyte-responsive active area, also referred to herein as a “sensing layer”) comprising an enzyme or enzyme system configured for assaying one or more analytes of interest. A counter electrode may be present in combination with the at least one working electrode, optionally in further combination with a reference electrode. Optionally, a second analyte-responsive active area, further optionally in combination with a second working electrode, may be located upon the sensor tail to facilitate detection of this analyte. Particular electrode configurations upon the sensor tail are described in more detail below in reference to FIGS. 2A-4. One or more enzymes in the active area may be covalently bonded to a polymer, according to various examples. Alternately, enzymes may be non-covalently associated within the active area, such as through encapsulation or physical entrainment. The one or more analytes may be monitored in any biological fluid of interest such as dermal fluid, interstitial fluid, plasma, blood, lymph, synovial fluid, cerebrospinal fluid, saliva, bronchoalveolar lavage, amniotic fluid, or the like. In particular examples, analyte sensors of the present disclosure may be adapted for assaying dermal fluid or interstitial fluid to determine analyte concentrations in vivo. It is to be appreciated, however, that the entirety of sensor control device may have one or more various configurations permitting full transplantation beneath tissue and into one or more body fluids for assaying one or more analytes of interest, without departing from the scope of the present disclosure.

Referring again to FIG. 1, sensor 104 may automatically forward data to reader device 120. For example, analyte concentration data may be communicated automatically and periodically, such as at a certain frequency as data is obtained or after a certain time period has passed, with the data being stored in a memory until transmittal (e.g. every minute, five minutes, or other predetermined time period), such as by BLUETOOTH® or BLUETOOTH® Low Energy protocols. Data associated with different analytes may be forwarded at the same frequency or different frequencies and/or using the same or different communication protocols. In other embodiments, sensor 104 may communicate with reader device 120 in a non-automatic manner and not according to a set schedule. For example, data may be communicated from sensor 104 using RFID technology when the sensor electronics are brought into communication range of reader device 120. Until communicated to reader device 120, data may remain stored in a memory of sensor 104. Thus, a user does not have to maintain close proximity to reader device 120 at all times, and can instead upload data at a convenient time, automatically or nonautomatically. In yet other examples, a combination of automatic and non-automatic data transfer may be implemented. For example, data transfer may continue on an automatic basis until reader device 120 is no longer in communication range of sensor 104.

An introducer may be present transiently to promote introduction of sensor 104 into a tissue. In illustrative examples, the introducer may comprise a needle or similar sharp, or a combination thereof. It is to be recognized that other types of introducers, such as sheaths or blades, may be present in alternative examples. More specifically, the needle or other introducer may transiently reside in proximity to sensor 104 prior to tissue insertion and then be withdrawn afterward. While present, the needle or other introducer may facilitate insertion of sensor 104 into a tissue by opening an access pathway for sensor 104 to follow. For example, the needle may facilitate penetration of the epidermis as an access pathway to the dermis to allow implantation of sensor 104 to take place, according to one or more examples. After opening the access pathway, the needle or other introducer may be withdrawn so that it does not represent 20 a sharps hazard. In illustrative examples, suitable needles may be solid or hollow, beveled or non-beveled, and/or circular or non-circular in cross-section. In more particular examples, suitable needles may be comparable in cross-sectional diameter and/or tip design to an acupuncture needle, which may have a cross-sectional diameter of about 250 microns. It is to be recognized, however, that suitable needles may have a larger or smaller cross-sectional diameter if needed for particular applications. For example, needles having a cross-sectional diameter ranging from about 300 microns to about 400 microns may be used.

In some examples, a tip of the needle (while present) may be angled over the terminus of sensor 104, such that the needle penetrates a tissue first and opens an access pathway for sensor 104. In other illustrative examples, sensor 104 may reside within a lumen or groove of the needle, with the needle similarly opening an access pathway for sensor 104. In either case, the needle may be subsequently withdrawn after facilitating sensor insertion.

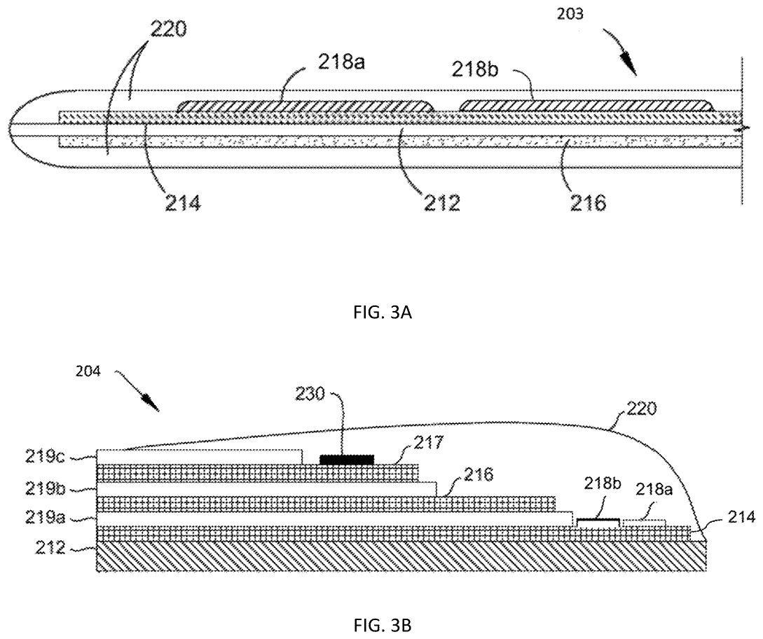

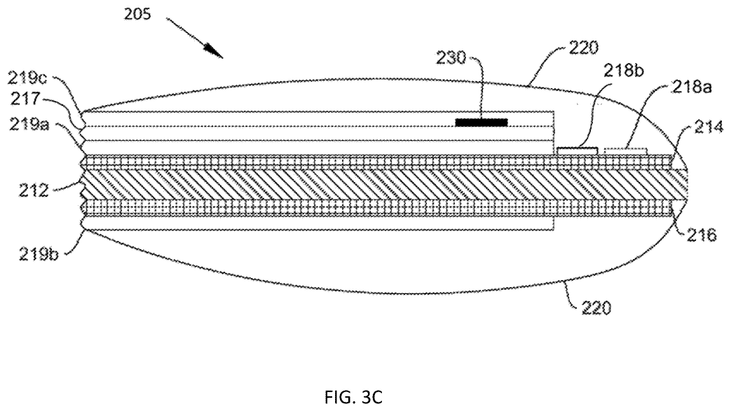

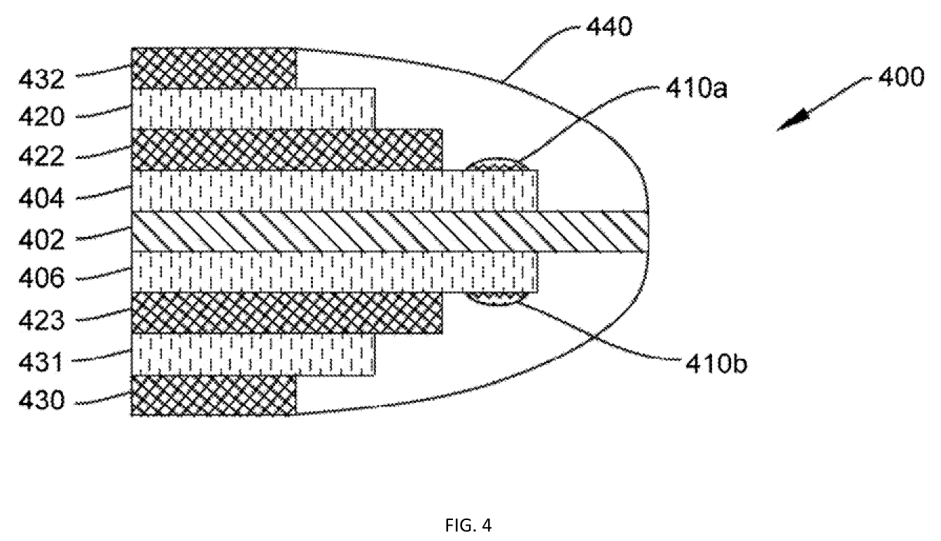

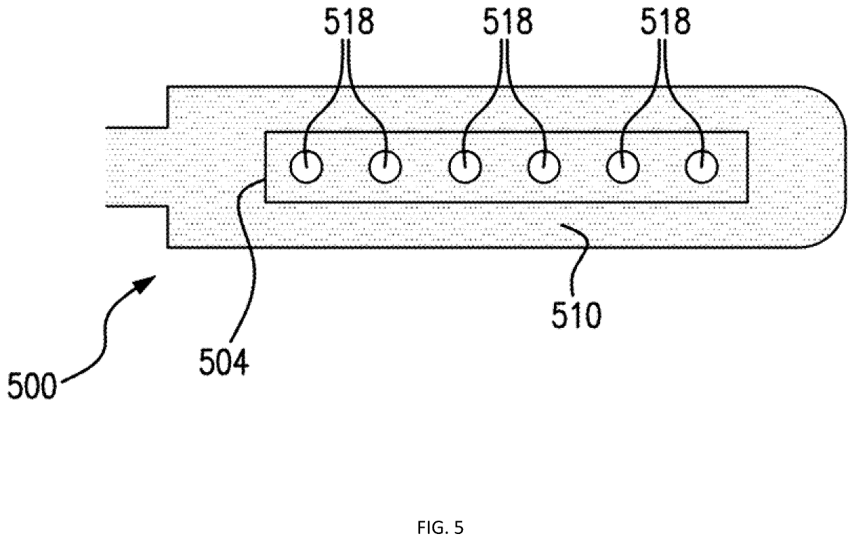

Sensor configurations featuring a single active area that is configured for detection of a corresponding single analyte may employ two-electrode or three-electrode detection motifs, as described further herein in reference to FIGS. 2A-2C. Sensor configurations featuring two different active areas for detection of separate analytes, either upon separate working electrodes or upon the same working electrode, are described separately thereafter in reference to FIGS. 3A-4. Sensor configurations having multiple working electrodes may be particularly advantageous for incorporating two different active areas within the same sensor tail, since the signal contribution from each active area may be determined more readily through separate interrogation of each working electrode. Each active area may be overcoated with a mass transport limiting membrane of the same or different composition.

In some embodiments, when a single working electrode is present in an analyte sensor, three-electrode sensor configurations may comprise a working electrode, a counter electrode, and a reference electrode. Related two-electrode sensor configurations may comprise a working electrode and a second electrode, in which the second electrode may function as both a counter electrode and a reference electrode (i.e. a counter/reference electrode). The various electrodes may be at least partially stacked (layered) upon one another and/or laterally spaced apart from one another upon the sensor tail. In any of the sensor configurations disclosed herein, the various electrodes may be electrically isolated from one another by a dielectric material or similar insulator.

Analyte sensors featuring multiple working electrodes may similarly comprise at least one additional electrode. When one additional electrode is present, the one additional electrode may function as a counter/reference electrode for each of the multiple working electrodes. When two additional electrodes are present, one of the additional electrodes may function as a counter electrode for each of the multiple working electrodes and the other of the additional electrodes may function as a reference electrode for each of the multiple working electrodes.

Any of the working electrode configurations described hereinafter may benefit from the further disclosure below directed to decreasing the availability of edge asperities of the working electrode upon the sensor tail.

FIG. 2A shows a diagram of an illustrative two-electrode analyte sensor configuration, which is compatible for use in the disclosure herein. As shown, analyte sensor 200 comprises substrate 212 disposed between working electrode 214 and counter/reference electrode 216. Alternately, working electrode 214 and counter/reference electrode 216 may be located upon the same side of substrate 212 with a dielectric material interposed in between (configuration not shown). Active area 218 is disposed as at least one layer upon at least a portion of working electrode 214. Active area 218 may comprise multiple spots or a single spot configured for detection of an analyte, as discussed further herein. Referring still to FIG. 2A, membrane 220 (referred to herein as the polymeric membrane) overcoats at least active area 218 and may optionally overcoat some or all of working electrode 214 and/or counter/reference electrode 216, or the entirety of analyte sensor 200, according to some examples. One or both faces of analyte sensor 200 may be overcoated with membrane 220. Membrane 220 may comprise one or more polymeric membrane materials having capabilities of limiting analyte flux to active area 218 (e.g. membrane 220 is a mass transport limiting membrane having some permeability for the analyte of interest). The composition and thickness of membrane 220 may vary to promote a desired analyte flux to active area 218, thereby providing a desired signal intensity and stability. Analyte sensor 200 may be operable for assaying an analyte by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations may be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 may then function as either a counter electrode or a reference electrode, and additional electrode 217 fulfills the other electrode function not otherwise accounted for. Working electrode 214 continues to fulfill its original function. Additional electrode 217 may be disposed upon either working electrode 214 or electrode 216, with a separating layer of dielectric material in between. For example, as depicted in FIG. 2B, dielectric layers 219a, 219b, and 219c separate electrodes 214, 216, and 217 from one another and provide electrical isolation. Alternately, at least one of electrodes 214, 216, and 217 may be located upon opposite faces of substrate 212, as shown in FIG. 2C. Thus, in some examples, electrode 214 (working electrode) and electrode 216 (counter electrode) may be located upon opposite faces of substrate 212, with electrode 217 (reference electrode) being located upon one of electrodes 214 or 216 and spaced apart therefrom with a dielectric material. Reference material layer 230 (e.g. Ag/AgCl) may be present upon electrode 217, with the location of reference material layer 230 not being limited to that depicted in FIGS. 2B and 2C. As with sensor 200 shown in FIG. 2A, active area 218 in analyte sensors 201 and 202 may comprise multiple spots or a single spot. Additionally, analyte sensors 201 and 202 may likewise be operable for assaying an analyte by any of coulometric, amperometric, voltammetric, or potentiometric electrochemical detection techniques. Like analyte sensor 200, membrane 220 (e.g. the polymeric membrane) may also overcoat active area 218, as well as other sensor components, in analyte sensors 201 and 202, thereby serving as a mass transport limiting membrane. Additional electrode 217 may be overcoated with membrane 220 in some examples. Membrane 220 may again be produced through dip coating or in situ photopolymerization and vary compositionally or be the same compositionally at different locations. Although FIGS. 2B and 2C have depicted all of electrodes 214, 216, and 217 as being overcoated with membrane 220, it is to be recognized that only working electrode 214 or active area 218 may be overcoated in some examples. Moreover, the thickness of membrane 220 at each of electrodes 214, 216, and 217 may be the same or different. As in two-electrode analyte sensor configurations (FIG. 2A), one or both faces of analyte sensors 201 and 202 may be overcoated with membrane 220 in the sensor configurations of FIGS. 2B and 2C, or the entirety of analyte sensors 201 and 202 may be overcoated. Accordingly, the three-electrode sensor configurations shown in FIGS. 2B and 2C should be understood as being non-limiting of the examples disclosed herein, with alternative electrode and/or layer configurations remaining within the scope of the present disclosure.

FIG. 3A shows an illustrative configuration for sensor 203 having a single working electrode with two different active areas disposed thereon. FIG. 3A is similar to FIG. 2A, except for the presence of two active areas upon working electrode 214: first active area 218a and second active area 218b, which are responsive to different analytes and are laterally spaced apart from one another upon the surface of working electrode 214. Active areas 218a and 218b may comprise multiple spots or a single spot configured for detection of each analyte. The composition of membrane 220 may vary or be compositionally the same at active areas 218a and 218b. First active area 218a and second active area 218b may be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first active area 218a and second active area 218b disposed thereon. FIGS. 3B and 3C are otherwise similar to FIGS. 2B and 2C and may be better understood by reference thereto. As with FIG. 3A, the composition of membrane 220 may vary or be compositionally the same at active areas 218a and 218b.

FIG. 4 shows a cross-sectional diagram of an illustrative analyte sensor configuration having two working electrodes, a reference electrode, and a counter electrode, which is compatible for use in the disclosure herein. As shown, analyte sensor 400 includes working electrodes 404 and 406 disposed upon opposite faces of substrate 402. First active area 410a is disposed upon the surface of working electrode 404, and second active area 410b is disposed upon the surface of working electrode 406. Counter electrode 420 is electrically isolated from working electrode 404 by dielectric layer 422, and reference electrode 431 is electrically isolated from working electrode 406 by dielectric layer 423. Outer dielectric layers 430 and 432 are positioned upon reference electrode 431 and counter electrode 420, respectively. Membrane 440 may overcoat at least active areas 410a and 410b, according to various examples, with other components of analyte sensor 400 or the entirety of analyte sensor 400 optionally being overcoated with membrane 440 as well. Again, membrane 440 may vary compositionally at active areas 410a and 410b, if needed, in order to afford suitable permeability values for differentially regulating the analyte flux at each location.

Alternative sensor configurations having multiple working electrodes and differing from the configuration shown in FIG. 4 may feature a counter/reference electrode instead of separate counter and reference electrodes, and/or feature layer and/or membrane arrangements varying from those expressly depicted. For example, the positioning of counter electrode 420 and reference electrode 431 may be reversed from that depicted in FIG. 4. In addition, working electrodes 404 and 406 need not necessarily reside upon opposing faces of substrate 302 in the manner shown in FIG. 4.

A carbon working electrode may suitably comprise the working electrode(s) in any of the analyte sensors disclosed herein. While carbon working electrodes are employed in electrochemical detection, use thereof in electrochemical sensing is not without difficulties. In particular, current related to an analyte of interest only results when an active area interacts with an analyte and transfers electrons to the portion of the carbon working electrode adjacent to the active area. Bodily fluid containing an analyte of interest also interacts with a carbon surface of the carbon working electrode not overcoated with an active area and does not contribute to the analyte signal, since there is no enzyme or enzyme system present at these locations to facilitate electron transfer from the analyte to the working electrode. Interferents may, however, undergo oxidation at portions of the working electrode lacking an active area and contribute background to the overall signal. Thus, carbon working electrodes with an extraneous (or “exposed”) carbon area upon the electrode surface do not meaningfully contribute to the analyte signal and may lead to contributory background signals in some cases. Other electrodes having an excessive surface area not directly detecting an analyte of interest may experience similar background signals and may be enhanced through modification of the disclosure herein.

Although various interferents may interact with the working electrode of the analyte sensors described herein, ascorbic acid is one example of an interferent commonly present in biological fluids that may generate a background signal at a carbon working electrode. For example, ascorbic acid oxidizes at the working electrode to produce dehydroascorbic acid. Various examples of the present disclosure will be described herein with reference to the interferent being ascorbic acid; however, it is to be understood that that the examples and analyte sensor configurations described herein are equally applicable to other interferents (electroactive species within a bodily fluid having an analyte of interest).

As provided above, the active area described herein may be a single sensing layer or a sensing layer having multiple sensing spots. Referring now to FIG. 5, illustrated is a top view of a carbon working electrode 500 having an active area 504 disposed thereon comprising multiple sensing spots 518. Only portions of carbon working electrode 500 comprising the sensing spots 518 contribute signal associated with an analyte of interest when the analyte interacts with the active area 504. Although carbon working electrode 500 shows six sensing spots 518 within the active area 504, it is to be appreciated that fewer or greater than six sensing spots 518 may be included upon carbon working electrode 500, without departing from the scope of the present disclosure. Extraneous carbon area 510 is not directly overlaid with sensing spots 518 and does not contribute signal associated with the analyte but may generate a background signal associated with one or more interferents. Accordingly, the oxidation of interferents at carbon working electrode 500 is proportional to the area of extraneous carbon area 510 available for interaction with the interferents. Indeed, the oxidation of ascorbic acid at carbon working electrode 500 scales roughly linearly with the area of available extraneous carbon area 510. As shown, the active area 504 is discontiguous and in the form of multiple sensing spots 518. As defined herein, the term “discontiguous,” and grammatical variants thereof, means that any single spot (sensing element) does not share an edge or boundary (e.g. is not touching) an adjacent spot.

### Active Area Sensing Chemistries

Active areas within any of the analyte sensors disclosed herein may comprise one or more analyte-responsive enzymes, either acting alone or in concert within an enzyme system. Any suitable analyte-responsive enzyme system known in the art may be used in the sensors of the present invention. In some embodiments, one or more enzymes may be covalently bonded to a polymer within the active area, as can one or more electron transfer agents located within the active area. Examples of suitable polymers within each active area may include poly(4-vinylpyridine) and poly(N-vinylimidazole) or a copolymer thereof, for example, in which quaternized pyridine and imidazole groups serve as a point of attachment for an electron transfer agent or enzyme(s). Other suitable polymers that may be present within the active area include, but are not limited to, those described in U.S. Pat. No. 6,605,200, incorporated herein by reference in its entirety, such as poly(acrylic acid), styrene/maleic anhydride copolymer, methylvinylether/maleic anhydride copolymer (GANTREZ polymer), poly(vinylbenzylchloride), poly(allylamine), poly lysine, poly(4-vinylpyridine) quaternized with carboxypentyl groups, and poly(sodium 4-styrene sulfonate). In some embodiments, the enzymes are bound to a redox polymer, or a polymer comprising a polymeric backbone, a crosslinker, and a transition metal complex. Suitable redox polymers are described in more detail in a separate section below.

Enzymes covalently bound to the polymer within the active areas that are capable of promoting analyte detection are not believed to be particularly limited. Suitable enzymes may include those capable of detecting glucose, lactate, ketones, creatinine, or the like. Any of these analytes may be detected in combination with one another in analyte sensors capable of detecting multiple analytes. Suitable enzymes and enzyme systems for detecting these analytes are described hereinafter.

In some examples, the analyte sensors may comprise a glucose-responsive active area comprising a glucose-responsive enzyme disposed upon the sensor tail. Suitable glucose-responsive enzymes may include, for example, glucose oxidase or a glucose dehydrogenase (e.g. pyrroloquinoline quinone (PQQ) or a cofactor-dependent glucose dehydrogenase, such as flavine adenine dinucleotide (FAD)-dependent glucose dehydrogenase or nicotinamide adenine dinucleotide (NAD)-dependent glucose dehydrogenase). Glucose oxidase and glucose dehydrogenase are differentiated by their ability to utilize oxygen as an electron acceptor when oxidizing glucose; glucose oxidase may utilize oxygen as an electron acceptor, whereas glucose dehydrogenases transfer electrons to natural or artificial electron acceptors, such as an enzyme cofactor. Glucose oxidase or glucose dehydrogenase may be used to promote detection. Both glucose oxidase and glucose dehydrogenase may be covalently bonded to a polymer comprising the glucose-responsive active area and exchange electrons with an electron transfer agent (e.g. an osmium (Os) complex or similar transition metal complex), which may also be covalently bonded to the polymer. Suitable electron transfer agents are described in further detail below. Glucose oxidase may directly exchange electrons with the electron transfer agent, whereas glucose dehydrogenase may utilize a cofactor to promote electron exchange with the electron transfer agent. FAD cofactor may directly exchange electrons with the electron transfer agent. NAD cofactor, in contrast, may utilize diaphorase to facilitate electron transfer from the cofactor to the electron transfer agent. Further details concerning glucose-responsive active areas incorporating glucose oxidase or glucose dehydrogenase, as well as glucose detection therewith, may be found in commonly owned U.S. Pat. No. 8,268,143, for example.

In some examples, the active areas of the present disclosure may be configured for detecting ketones. Additional details concerning enzyme systems responsive to ketones may be found in commonly owned U.S. patent application Ser. No. 16/774,835 entitled “Analyte Sensors and Sensing Methods Featuring Dual Detection of Glucose and Ketones,” filed on Jan. 28, 2020, and published as U.S. Patent Application Publication 2020/0237275, the contents of which is incorporated in its entirety herein. In such systems, β-hydroxybutyrate serves as a surrogate for ketones formed in vivo, which undergoes a reaction with an enzyme system comprising 3-hydroxybutyrate dehydrogenase (HBDH) and diaphorase to facilitate ketones detection within a ketones-responsive active area disposed upon the surface of at least one working electrode, as described further herein. Within the ketones-responsive active area, hydroxybutyrate dehydrogenase may convert β-hydroxybutyrate and oxidized nicotinamide adenine dinucleotide (NAD+) into acetoacetate and reduced nicotinamide adenine dinucleotide (NADH), respectively. It is to be understood that the term “nicotinamide adenine dinucleotide (NAD)” includes a phosphate-bound form of the foregoing enzyme cofactors. That is, use of the term “NAD” herein refers to both NAD+ phosphate and NADH phosphate, specifically a diphosphate linking the two nucleotides, one containing an adenine nucleobase and the other containing a nicotinamide nucleobase. The NAD+/NADH enzyme cofactor aids in promoting the concerted enzymatic reactions disclosed herein. Once formed, NADH may undergo oxidation under diaphorase mediation, with the electrons transferred during this process providing the basis for ketones detection at the working electrode. Thus, there is a 1:1 molar correspondence between the amount of electrons transferred to the working electrode and the amount of β-hydroxybutyrate converted. Transfer of the electrons to the working electrode may take place under further mediation of an electron transfer agent, such as an osmium (Os) compound or similar transition metal complex, as described in additional detail below. Albumin may further be present as a stabilizer within the active area. The hydroxybutyrate dehydrogenase and the diaphorase may be covalently bonded to a polymer comprising the ketones-responsive active area. The NAD+ may or may not be covalently bonded to the polymer, but if the NAD+ is not covalently bonded, it may be physically retained within the ketones-responsive active area, such as with a mass transport limiting membrane overcoating the ketones-responsive active area, wherein the mass transport limiting membrane is also permeable to ketones.

Other suitable chemistries for enzymatically detecting ketones may be utilized in accordance with the examples of the present disclosure. For example, β-hydroxybutyrate dehydrogenase (HBDH) may again convert β-hydroxybutyrate and NAD+ into acetoacetate and NADH, respectively. Instead of electron transfer to the working electrode being completed by diaphorase and a suitable redox mediator, the reduced form of NADH oxidase (NADHOx (Red)) undergoes a reaction to form the corresponding oxidized form (NADHOx (Ox)). NADHOx (Red) may then reform through a reaction with molecular oxygen to produce superoxide, which may undergo subsequent conversion to hydrogen peroxide under superoxide dismutase (SOD) mediation. The hydrogen peroxide may then undergo oxidation at the working electrode to provide a signal that may be correlated to the amount of ketones that were initially present. The SOD may be covalently bonded to a polymer in the ketones-responsive active area, according to various examples. The β-hydroxybutyrate dehydrogenase and the NADH oxidase may be covalently bonded to a polymer in the ketones-responsive active area, and the NAD+ may or may not be covalently bonded to a polymer in the ketones-responsive active area. If the NAD+ is not covalently bonded, it may be physically retained within the ketones-responsive active area, with a membrane polymer promoting retention of the NAD+ within the ketones-responsive active area. There is again a 1:1 molar correspondence between the amount of electrons transferred to the working electrode and the amount of β-hydroxybutyrate converted, thereby providing the basis for ketones detection.

Another enzymatic detection chemistry for ketones may utilize β-hydroxybutyrate dehydrogenase (HBDH) to convert β-hydroxybutyrate and NAD+ into acetoacetate and NADH, respectively. The electron transfer cycle in this case is completed by oxidation of NADH by 1,10-phenanthroline-5,6-dione to reform NAD+, wherein the 1,10-phenanthroline-5,6-dione subsequently transfers electrons to the working electrode. The 1,10-phenanthroline-5,6-dione may or may not be covalently bonded to a polymer within the ketones-responsive active area. The β-hydroxybutyrate dehydrogenase may be covalently bonded to a polymer in the ketones responsive active area, and the NAD+ may or may not be covalently bonded to a polymer in the ketones-responsive active area. Inclusion of an albumin in the active area may provide a surprising improvement in response stability. A suitable membrane polymer may promote retention of the NAD+ within the ketones-responsive active area. There is again a 1:1 molar correspondence between the amount of electrons transferred to the working electrode and the amount of β-hydroxybutyrate converted, thereby providing the basis for ketones detection.

In some examples, the analyte sensors may further comprise a creatinine-responsive active area comprising an enzyme system that operates in concert to facilitate detection of creatinine. Creatinine may react reversibly and hydrolytically in the presence of creatinine amidohydrolase (CNH) to form creatine. Creatine, in turn, may undergo catalytic hydrolysis in the presence of creatine amidohydrolase (CRH) to form sarcosine. Neither of these reactions produces a flow of electrons (e.g. oxidation or reduction) to provide a basis for electrochemical detection of the creatinine. The sarcosine produced via hydrolysis of creatine may undergo oxidation in the presence of the oxidized form of sarcosine oxidase (SOX-ox) to form glycine and formaldehyde, thereby generating the reduced form of sarcosine oxidase (SOX-red) in the process. Hydrogen peroxide also may be generated in the presence of oxygen. The reduced form of sarcosine oxidase, in turn, may then undergo re-oxidation in the presence of the oxidized form of an electron transfer agent (e.g. an Os(III) complex), thereby producing the corresponding reduced form of the electron transfer agent (e.g. an Os(II) complex) and delivering a flow of electrons to the working electrode.

Oxygen may interfere with the concerted sequence of reactions used to detect creatinine in accordance with the disclosure above. Specifically, the reduced form of sarcosine oxidase may undergo a reaction with oxygen to reform the corresponding oxidized form of this enzyme but without exchanging electrons with the electron transfer agent. Although the enzymes all remain active when the reaction with oxygen occurs, no electrons flow to the working electrode. Without being bound by theory or mechanism, the competing reaction with oxygen is believed to result from kinetic effects. That is, oxidation of the reduced form of sarcosine oxidase with oxygen is believed to occur faster than does oxidation promoted by the electron transfer agent. Hydrogen peroxide is also formed in the presence of the oxygen.

The desired reaction pathway for facilitating detection of creatinine may be encouraged by including an oxygen scavenger in proximity to the enzyme system. Various oxygen scavengers and dispositions thereof may be suitable, including oxidase enzymes such as glucose oxidase. Small molecule oxygen scavengers may also be suitable, but they may be fully consumed before the sensor lifetime is otherwise fully exhausted. Enzymes, in contrast, may undergo reversible oxidation and reduction, thereby affording a longer sensor lifetime. By discouraging oxidation of the reduced form of sarcosine oxidase with oxygen, the slower electron exchange reaction with the electron transfer agent may occur, thereby allowing production of a current at the working electrode. The magnitude of the current produced is proportional to the amount of creatinine that was initially reacted.

The oxygen scavenger used for encouraging the desired reaction may be an oxidase enzyme in any example of the present disclosure. Any oxidase enzyme may be used to promote oxygen scavenging in proximity to the enzyme system, provided that a suitable substrate for the enzyme is also present, thereby providing a reagent for reacting with the oxygen in the presence of the oxidase enzyme. Oxidase enzymes that may be suitable for oxygen scavenging in the present disclosure include, but are not limited to, glucose oxidase, lactate oxidase, xanthine oxidase, and the like. Glucose oxidase may be a particularly desirable oxidase enzyme to promote oxygen scavenging due to the ready availability of glucose in various bodily fluids. Reaction 1 below shows the enzymatic reaction promoted by glucose oxidase to afford oxygen clearing.

$$β - D - glucose + 02 = D - glucono - 1 , 5 - lactone + H 202 \qquad Reaction 1$$

The concentration of available lactate in vivo is lower than that of glucose, but still sufficient to promote oxygen scavenging.

Oxidase enzymes, such as glucose oxidase, may be positioned in any location suitable to promote oxygen scavenging in the analyte sensors disclosed herein. Glucose oxidase, for example, may be positioned upon the sensor tail such that the glucose oxidase is functional and/or non-functional for promoting glucose detection. When non-functional for promoting glucose detection, the glucose oxidase may be positioned upon the sensor tail such that electrons produced during glucose oxidation are precluded from reaching the working electrode, such as through electrically isolating the glucose oxidase from the working electrode.

Additional details concerning enzyme systems responsive to creatinine may be found in commonly owned U.S. patent application Ser. No. 16/774,835 entitled “Analyte Sensors and Sensing Methods for Detecting Creatinine,” filed on Sep. 25, 2019, and published as U.S. Patent Application Publication 2020/0237275, which is incorporated herein by reference in its entirety.

In some examples, the analyte sensors may comprise a lactate-responsive active area comprising a lactate-responsive enzyme disposed upon the sensor tail. Suitable lactate-responsive enzymes may include, for example, lactate oxidase. Lactate oxidase or other lactate-responsive enzymes may be covalently bonded to a polymer comprising the lactate responsive active area and exchange electrons with an electron transfer agent (e.g. an osmium (Os)) complex or similar transition metal complex), which may also be covalently bonded to the polymer. Suitable electron transfer agents are described in further detail below. An albumin, such as human serum albumin, may be present in the lactate-responsive active area to stabilize the sensor response, as described in further detail in commonly owned U.S. Patent Application Publication 20190320947, which is incorporated herein by reference in its entirety. Lactate levels may vary in response to numerous environmental or physiological factors including, for example, eating, stress, exercise, sepsis or septic shock, infection, hypoxia, presence of cancerous tissue, or the like.

In some examples, the analyte sensors may comprise an active area responsive to pH. Suitable analyte sensors configured for determining pH are described in commonly owned U.S. Patent Application Publication 20200060592, which is incorporated herein by reference. Such analyte sensors may comprise a sensor tail comprising a first working electrode and a second working electrode, wherein a first active area located upon the first working electrode comprises a substance having pH-dependent oxidation-reduction chemistry, and a second active area located upon the second working electrode comprises a substance having oxidation-reduction chemistry that is substantially invariant with pH. By obtaining a difference between the first signal and the second signal, the difference may be correlated to the pH of a fluid to which the analyte sensor is exposed.

Two different types of active areas may be located upon a single working electrode, such as the carbon working electrodes discussed above, and spaced apart from one another. Each active area may have an oxidation-reduction potential, wherein the oxidation reduction potential of the first active area is sufficiently separated from the oxidation-reduction potential of the second active area to allow independent production of a signal from one of the active areas. By way of non-limiting example, the oxidation-reduction potentials may differ by at least about 100 mV, or by at least about 150 mV, or by at least about 200 mV. The upper limit of the separation between the oxidation-reduction potentials is dictated by the working electrochemical window in vivo. By having the oxidation-reduction potentials of the two active areas sufficiently separated in magnitude from one another, an electrochemical reaction may take place within one of the two active areas (i.e. within the first active area or the second active area) without substantially inducing an electrochemical reaction within the other active area. Thus, a signal from one of the first active area or the second active area may be independently produced at or above its corresponding oxidation-reduction potential (the lower oxidation-reduction potential) but below the oxidation-reduction potential of the other active area. A different signal may allow the signal contribution from each analyte to be resolved.

Some or all examples of analyte sensors disclosed herein may feature one or more active areas located upon the surface of at least one working electrode, where the active areas detect the same or different analytes.

An electron transfer agent may be present in any of the active areas disclosed herein. Suitable electron transfer agents may facilitate conveyance of electrons to the adjacent working electrode after one or more analytes undergoes an enzymatic oxidation-reduction reaction within the corresponding active area, thereby generating an electron flow that is indicative of the presence of a particular analyte. The amount of current generated is proportional to the quantity of analyte that is present. Depending on the sensor configuration used, the electron transfer agents in active areas responsive to different analytes may be the same or different. For example, when two different active areas are disposed upon the same working electrode, the electron transfer agent within each active area may be different (e.g. chemically different such that the electron transfer agents exhibit different oxidation-reduction potentials). When multiple working electrodes are present, the electron transfer agent within each active area may be the same or different, since each working electrode may be interrogated separately.

Suitable electron transfer agents may include electroreducible and electrooxidizable ions, complexes or molecules (e.g. quinones) having oxidation-reduction potentials that are a few hundred millivolts above or below the oxidation-reduction potential of the standard calomel electrode (SCE). According to some examples, suitable electron transfer agents may include low-potential osmium complexes, such as those described in U.S. Pat. Nos. 6,134,461 and 6,605,200, which are incorporated herein by reference in their entirety. Additional examples of suitable electron transfer agents include those described in U.S. Pat. Nos. 6,736,957, 7,501,053 and 7,754,093, the disclosures of each of which are incorporated herein by reference in their entirety. Other suitable electron transfer agents may comprise metal compounds or complexes of ruthenium, osmium, iron (e.g. polyvinylferrocene or hexacyanoferrate), or cobalt, including metallocene compounds thereof, for example. Suitable ligands for the metal complexes may also include, for example, bidentate or higher denticity ligands such as, for example, bipyridine, biimidazole, phenanthroline, or pyridyl(imidazole). Other suitable bidentate ligands may include, for example, amino acids, oxalic acid, acetylacetone, diaminoalkanes, or o-diaminoarenes. Any combination of monodentate, bidentate, tridentate, tetradentate, or higher denticity ligands may be present in a metal complex to achieve a full coordination sphere.

In some embodiments, electron transfer agents are covalently bound to a polymer, such as to a polymer membrane of the active area. A polymer comprising electron transfer reagents (e.g. a polymer wherein electron transfer agents are covalently bound to the polymer) is also referred to herein as a “redox polymer”, and is described in more detail below. Any of the electron transfer agents disclosed herein may comprise suitable functionality to promote covalent bonding to the polymer within the active areas. Suitable examples of polymer-bound electron transfer agents may include those described in U.S. Pat. Nos. 8,444,834, 8,268,143 and 6,605,201, the disclosures of which are incorporated herein by reference in their entirety. Suitable polymers for inclusion in the active areas may include, but are not limited to, polyvinylpyridines (e.g. poly(4-vinylpyridine)), polyvinylimidazoles (e.g. poly(1-vinylimidazole)), or any copolymer thereof. Illustrative copolymers that may be suitable for inclusion in the active areas include those containing monomer units such as styrene, acrylamide, methacrylamide, or acrylonitrile, for example. When two or more different active areas are present, the polymer within each active area may be the same or different.

Covalent bonding of the electron transfer agent to a polymer within an active area may take place by polymerizing a monomer unit bearing a covalently bonded electron transfer agent, or the electron transfer agent may be reacted with the polymer separately after the polymer has already been synthesized. A bifunctional spacer may covalently bond the electron transfer agent to the polymer within the active area, with a first functional group being reactive with the polymer (e.g. a functional group capable of quaternizing a pyridine nitrogen atom or an imidazole nitrogen atom) and a second functional group being reactive with the electron transfer agent (e.g. a functional group that is reactive with a ligand coordinating a metal ion).

Similarly, one or more of the enzymes within the active areas may be covalently bonded to a polymer comprising or covering an active area (e.g. a membrane polymer coating the sensing layer). When an enzyme system comprising multiple enzymes is present in a given active area, all of the multiple enzymes may be covalently bonded to the polymer in some examples, and in other examples, only a portion of the multiple enzymes may be covalently bonded to the polymer. For example, one or more enzymes comprising an enzyme system may be covalently bonded to the polymer and at least one enzyme may be non-covalently associated with the polymer, such that the non-covalently bonded enzyme is physically entrained within the polymer. Covalent bonding of the enzyme(s) to the polymer in a given active area (e.g. to the membrane polymer coating the active area) may take place via a crosslinker introduced with a suitable crosslinking agent. Suitable crosslinking agents for reaction with free amino groups in the enzyme (e.g. with the free side chain amine in lysine) may include crosslinking agents such as, for example polyethylene glycol diglycidyl ether (PEGDGE) or other polyepoxides, cyanuric chloride, N-hydroxysuccinimide, imidoesters, epichlorohydrin, or derivatized variants thereof. Suitable crosslinking agents for reaction with free carboxylic acid groups in the enzyme may include, for example, carbodiimides. The crosslinking of the enzyme to the polymer is generally intermolecular, but can be intramolecular in some embodiments. In particular examples, all of the enzymes within a given active area may be covalently bonded to a polymer.

The electron transfer agent and/or the enzyme(s) may be associated with the polymer in an active area through means other than covalent bonding as well. In some examples, the electron transfer agent and/or the enzyme(s) may be ionically or coordinatively associated with the polymer. For example, a charged polymer may be ionically associated with an oppositely charged electron transfer agent or enzyme(s). In still other examples, the electron transfer agent and/or the enzyme(s) may be physically entrained within the polymer without being bonded thereto. Physically entrained electron transfer agents and/or enzyme(s) may still suitably interact with a fluid to promote analyte detection without being substantially leached from the active areas.

The polymer within or coating the active area may be chosen such that outward diffusion of NAD+ or another cofactor not covalently bound to the polymer is limited. Limited outward diffusion of the cofactor may promote a reasonable sensor lifetime (days to weeks) while still allowing sufficient inward analyte diffusion to promote detection.

In some examples, a stabilizer may be incorporated into the active area of the analyte sensors described herein to improve the functionality of the sensors and achieve desired sensitivity and stability. Such stabilizers may include an antioxidant and/or companion protein to stabilize the enzyme, for instance. Examples of suitable stabilizers may include, but are not limited to serum albumin (e.g. human or bovine serum albumin or other compatible albumin), catalase, other enzyme antioxidants, and the like, and any combination thereof. The stabilizers may be conjugated or non-conjugated.

### Redox Polymers

In some embodiments, the sensor comprises a redox polymer. Exemplary redox polymers are described in U.S. Pat. Nos. 8,444,834 and 8,268,143, the entire contents of each of which are incorporated herein by reference for all purposes. In some embodiments, the redox polymer comprises polymeric backbone, a crosslinker, and a transition metal complex. In some embodiments, the redox polymer comprises a poly(vinylpyridine)-based polymer (e.g. a poly(vinylpyridine)-based polymeric backbone), a crosslinker, and a transition metal complex. In some embodiments, the transition metal complex comprises osmium. Such a redox polymer is referred to herein as an osmium-decorated polymer.

In some embodiments, the redox polymer comprises a transition metal complex having one or more of the following characteristics: redox potentials in a particular range, the ability to exchange electrons rapidly with electrodes, the ability to rapidly transfer electrons to or rapidly accept electrons from an enzyme to accelerate the kinetics of electrooxidation or electroreduction of an analyte in the presence of an enzyme or another analyte-specific redox catalyst. For example, a redox mediator may accelerate the electrooxidation of glucose in the presence of a analyte response dehydrogenase optionally complexed with a co-factor, such as GDH optionally complexed with a co-factor including FAD, a process that can be useful for the selective assay of glucose in the presence of other electrochemically oxidizable species.

Compounds having the formula 1 are examples of transition metal complexes of the embodiments of the invention.

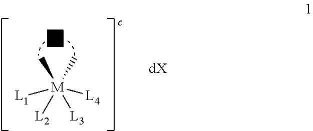

M is a transition metal and is typically iron, cobalt, ruthenium, osmium, or vanadium. Ruthenium and osmium are particularly suitable for redox mediators. In exemplary embodiments, the transition metal is osmium.
L is a bidentate ligand containing at least one imidazole ring. One example of L is a 2,2′-biimidazole having the following structure 2:

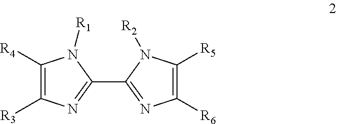

R₁ and R₂ are substituents attached to two of the 2,2′-biimidazole nitrogens and are independently substituted or unsubstituted alkyl, alkenyl, or aryl groups. Generally, R₁ and R₂ are unsubstituted C1 to C12 alkyls. Typically, R₁ and R₂ are unsubstituted C1 to C4 alkyls. In some embodiments, both R₁ and R₂ are methyl.
R₃, R₄, R₅, and R₆ are substituents attached to carbon atoms of the 2,2′-biimidazole and are independently —H, —F, —Cl, —Br, —I, —NO₂, —CN, —CO₂H, —SO₃H, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl. Alternatively, R₃ and R₄ in combination or R₅ and R₆ in combination independently form a saturated or unsaturated 5- or 6-membered ring. An example of this is a 2,2′-bibenzoimidazole derivative. Typically, the alkyl and alkoxy portions are C1 to C12. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group. Generally, R₃, R₄, R₅, and R₆ are independently —H or unsubstituted alkyl groups. Typically, R₃, R₄, R₅, and R₆ are —H or unsubstituted C1 to C12 alkyls. In some embodiments, R₃, R₄, R₅, and R₆ are all —H.

Another example of L is a 2-(2-pyridyl)imidazole having the following structure 3:

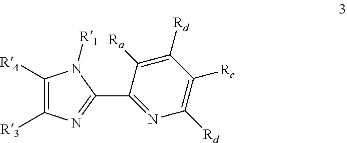

R′₁ is a substituted or unsubstituted aryl, alkenyl, or alkyl. Generally, R′₁ is a substituted or unsubstituted C1-C12 alkyl. R′₁ is typically methyl or a C1-C12 alkyl that is optionally substituted with a reactive group.
R′₃, R′₄, Rₐ, R_{b}, R_{c}, and R_{d} are independently —H, —F, —Cl, —Br, —I, —NO₂, —CN, —CO₂H, —SO₃H, —NHNH₂, —SH, alkoxylcarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxylamino, alkylthio, alkenyl, aryl, or alkyl. Alternatively, R_{c} and R_{d} in combination or R′₃ and R′₄ in combination can form a saturated or unsaturated 5- or 6-membered ring. Typically, the alkyl and alkoxy portions are C1 to C12. The alkyl or aryl portions of any of the substituents are optionally substituted by F, Cl, Br, I, alkylamino, dialkylamino, trialkylammonium (except on aryl portions), alkoxy, alkylthio, aryl, or a reactive group. Generally, R′₃, R′₄, Rₐ, R_{b}, R_{c}, and R_{d} are independently —H or unsubstituted alkyl groups. Typically, Rₐ and R_{c} are —H and R′₃, R′₄, R_{b}, and R_{d} are H or methyl.
c is an integer indicating the charge of the complex. Generally, c is an integer selected from −1 to −5 or +1 to +5 indicating a positive or negative charge. For a number of osmium complexes, c is +2 or +3.
X represents counter ion(s). Examples of suitable counter ions include anions, such as halide (e.g. fluoride, chloride, bromide or iodide), sulfate, phosphate, hexafluorophosphate, and tetrafluoroborate, and cations (in some embodiments, monovalent cations), such as lithium, sodium, potassium, tetralkylammonium, and ammonium. In an embodiment of an embodiment of the invention, X is a halide, such as chloride. The counter ions represented by X are not necessarily all the same.
d represents the number of counter ions and is typically from 1 to 5.
L₁, L₂, L₃ and L₄ are ligands attached to the transition metal via a coordinative bond. L₁, L₂, L₃ and L₄ can be monodentate ligands or, in any combination, bi-, ter-, or tetradentate ligands For example, L₁, L₂, L₃ and L₄ can combine to form two bidentate ligands such as, for example, two ligands selected from the group of substituted and unsubstituted 2,2′-biimidazoles, 2-(2-pyridyl)imidizoles, and 2,2′-bipyridines.

The term “alkyl” includes, for example, linear or branched, saturated aliphatic hydrocarbons. Examples of alkyl groups include methyl, ethyl, n-propyl, isopropyl, n-butyl, tert-butyl and the like. Unless otherwise noted, the term “alkyl” includes, for example, both alkyl and cycloalkyl groups.

The term “alkoxy” refers to, for example, an alkyl group joined to the remainder of the structure by an oxygen atom. Examples of alkoxy groups include methoxy, ethoxy, n-propoxy, isopropoxy, butoxy, tert-butoxy, and the like. In addition, unless otherwise noted, the term ‘alkoxy’ includes, for example, both alkoxy and cycloalkoxy groups.

The term “alkenyl” refers to, for example, an unsaturated, linear or branched aliphatic hydrocarbon having at least one carbon-carbon double bond. Examples of alkenyl groups include ethenyl, 1-propenyl, 2-propenyl, 1-butenyl, 2-methyl-1-propenyl, and the like. A “substituted” functional group (e.g. substituted alkyl, alkenyl, or alkoxy group) includes at least one substituent selected from the following: halogen, alkoxy, mercapto, aryl, alkoxycarbonyl, alkylaminocarbonyl, dialkylaminocarbonyl, —OH, —NH₂, alkylamino, dialkylamino, trialkylammonium, alkanoylamino, arylcarboxamido, hydrazino, alkylthio, alkenyl, and reactive groups.

Examples of other L₁, L₂, L₃ and L₄ combinations of the transition metal complex include:
(A) L₁ is a monodentate ligand and L₂, L₃ and L₄ in combination form a terdentate ligand;
(B) L₁ and L₂ in combination are a bidentate ligand, and L₃ and L₄ are the same or different monodentate ligands;
(C) L₁ and L₂ in combination, and L₃ and L₄ in combination form two independent bidentate ligands which can be the same or different; and
(D) L₁, L₂, L₃ and L₄ in combination form a tetradentate ligand.

Examples of suitable monodentate ligands include, but are not limited to, —F, —Cl, —Br, —I, —CN, —SCN, —OH, —H₂O, —NH₃, alkylamine, dialkylamine, trialkylamine, alkoxy or heterocyclic compounds. The alkyl or aryl portions of any of the ligands are optionally substituted by —F, —Cl, —Br, —I, alkylamino, dialkylamino, trialkylammonium (except on aryl portions), alkoxy, alkylthio, aryl, or a reactive group. Any alkyl portions of the monodentate ligands generally contain 1 to 12 carbons. More typically, the alkyl portions contain 1 to 6 carbons. In other embodiments, the monodentate ligands are heterocyclic compounds containing at least one nitrogen, oxygen, or sulfur atom. Examples of suitable heterocyclic monodentate ligands include imidazole, pyrazole, oxazole, thiazole, pyridine, pyrazine and derivatives thereof. Suitable heterocyclic monodentate ligands include substituted and unsubstituted imidazole and substituted and unsubstituted pyridine having the following general formulas 4 and 5, respectively:

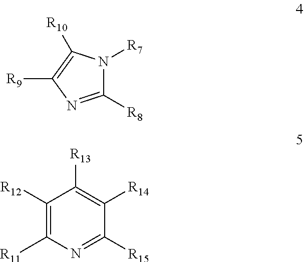

With regard to formula 4, R₇ is generally a substituted or unsubstituted alkyl, alkenyl, or aryl group. Typically, R₇ is a substituted or unsubstituted C1 to C12 alkyl or alkenyl. The substitution of inner coordination sphere chloride anions by imidazoles does not typically cause a large shift in the redox potential in the oxidizing direction, differing in this respect from substitution by pyridines, which typically results in a large shift in the redox potential in the oxidizing direction.

R₈, R₉ and R₁₀ are independently —H, —F, —Cl, —Br, —I, —NO₂, —CN, —CO₂H, —SO₃H, —NHNH₂, —SH, aryl, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl. Alternatively, R₉ and R₁₀, in combination, form a fused 5 or 6-membered ring that is saturated or unsaturated. The alkyl portions of the substituents generally contain 1 to 12 carbons and typically contain 1 to 6 carbon atoms. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group. In some embodiments, R₈, R₉ and R₁₀ are —H or substituted or unsubstituted alkyl. In an embodiment of an embodiment of the invention, R₈, R₉ and R₁₀ are H.

With regard to Formula 5, R₁₁, R₁₂, R₁₃, R₁₄ and R₁₅ are independently —H, —F, —Cl, —Br, —I, —NO₂, —CN, —CO₂H, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxyamino, alkylthio, alkenyl, aryl, or alkyl. The alkyl or aryl portions of any of the substituents are optionally substituted by —F, —Cl, —Br, —I, alkylamino, dialkylamino, trialkylammonium (except for aryl portions), alkoxy, alkylthio, aryl, or a reactive group. Generally, R₁₁, R₁₂, R₁₃, R₁₄ and R₁₅ are H, methyl, C1-C2 alkoxy, C1-C2 alkylamino, C2-C4 dialkylamino, or a C1-C6 lower alkyl substituted with a reactive group. One example includes R₁₁ and R₁₅ as —H, R₁₂ and R₁₄ as the same and —H or methyl, and R₁₃ as —H, C1 to C12 alkoxy, —NH₂, C1 to C12 alkylamino, C2 to C24 dialkylamino, hydrazino, C1 to C12 alkylhydrazino, hydroxylamino, C1 to C12 alkoxyamino, C1 to C12 alkylthio, or C1 to C12 alkyl. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group.

A “reactive group” is a functional group of a molecule that is capable of reacting with another compound to couple at least a portion of that other compound to the molecule. Exemplary reactive groups include, but are not limited to, carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

Examples of suitable bidentate ligands include, but are not limited to, amino acids, oxalic acid, acetylacetone, diaminoalkanes, ortho-diaminoarenes, 2,2′-biimidazole, 2,2′-bioxazole, 2,2′-bithiazole, 2-(2-pyridyl)imidazole, and 2,2′-bipyridine and derivatives thereof. Particularly suitable bidentate ligands for redox mediators include substituted and unsubstituted 2,2′-biimidazole, 2-(2-pyridyl)imidazole and 2,2′-bipyridine. The substituted 2,2′ biimidazole and 2-(2-pyridyl)imidazole ligands can have the same substitution patterns described above for the other 2,2′-biimidazole and 2-(2-pyridyl)imidazole ligand. A 2,2′-bipyridine ligand has the following general formula 6:

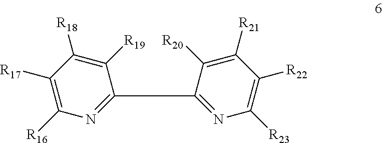

R₁₆, R₁₇, R₁₈, R₁₉, R₂₀, R₂₁, R₂₂ and R₂₃ are independently —H, —F, —Cl, —Br, —I, —NO₂, —CN, —CO₂H, —SO₃H, —NHNH₂, —SH, aryl, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxylamino, alkylthio, alkenyl, or alkyl. Typically, the alkyl and alkoxy portions are C1 to C12. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group.

Specific examples of suitable combinations of R₁₆, R₁₇, R₁₈, R₁₉, R₂₀, R₂₁, R₂₂ and R₂₃ include R₁₆ and R₂₃ as H or methyl; R₁₇ and R₂₂ as the same and H or methyl; and R₁₉ and R₂₀ as the same and —H or methyl. An alternative combination is where one or more adjacent pairs of substituents R₁₆ and R₁₇, on the one hand, and R₂₂ and R₂₃, on the other hand, independently form a saturated or unsaturated 5- or 6-membered ring. Another combination includes R₁₉ and R₂₀ forming a saturated or unsaturated five or six membered ring.

Another combination includes R₁₆, R₁₇, R₁₉, R₂₀, R₂₂ and R₂₃ as the same and —H and R₁₈ and R₂₁ as independently —H, alkoxy, —NH₂, alkylamino, dialkylamino, alkylthio, alkenyl, or alkyl. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group. As an example, R₁₈ and R₂₁ can be the same or different and are —H, C1-C6 alkyl, C1-C6 amino, C1 to C12 alkylamino, C2 to C12 dialkylamino, C1 to C12 alkylthio, or C1 to C12 alkoxy, the alkyl portions of any of the substituents are optionally substituted by a —F, —Cl, —Br, —I, aryl, C2 to C12 dialkylamino, C3 to C18 trialkylammonium, C1 to C6 alkoxy, C1 to C6 alkylthio or a reactive group.

Examples of suitable terdentate ligands include, but are not limited to, diethylenetriamine, 2,2′,2″-terpyridine, 2,6-bis(N-pyrazolyl)pyridine, and derivatives of these compounds. 2,2′,2″-terpyridine and 2,6-bis(N-pyrazolyl)pyridine have the following general formulas 7 and 8 respectively:

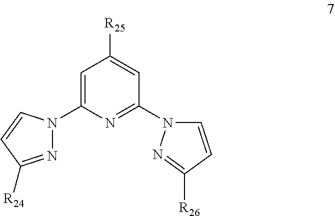

In some embodiments, the transition metal complex is a tridentate mediator comprising a similar structure as shown above, with comprising two five membered imidazole rings linked to a central pyridine through the 2′-carbon.

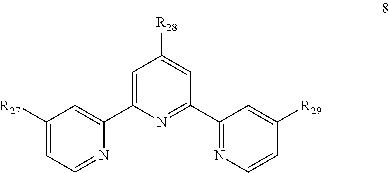

With regard to formula 7, R₂₄, R₂₅ and R₂₆ are independently —H or substituted or unsubstituted C1 to C12 alkyl. Typically, R₂₄, R₂₅ and R₂₆ are —H or methyl and, in some embodiments, R₂₄ and R₂₆ are the same and are —H. Other substituents at these or other positions of the compounds of formulas 7 and 8 can be added.

With regard to formula 8, R₂₇, R₂₈ and R₂₉ are independently —H, —F, —Cl, —Br, —I, —NO₂, —CN, —CO₂H, —SO₃H, —NHNH₂, —SH, alkoxycarbonyl, alkylaminocarbonyl, dialkylaminocarbonyl, —OH, alkoxy, —NH₂, alkylamino, dialkylamino, alkanoylamino, arylcarboxamido, hydrazino, alkylhydrazino, hydroxylamino, alkoxylamino, alkylthio, alkenyl, aryl, or alkyl. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group. Typically, the alkyl and alkoxy groups are C1 to C12 and, in some embodiments, R₂₇ and R₂₉ are the same and are —H.

Examples of suitable tetradentate ligands include, but are not limited to, triethylenetriamine, ethylenediaminediacetic acid, tetraaza macrocycles and similar compounds as well as derivatives thereof.

Examples of suitable transition metal complexes are illustrated using Formula 9 and 10:

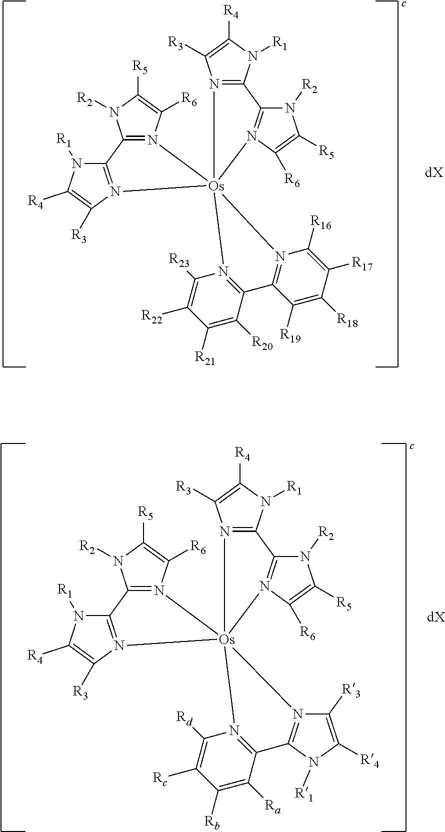

With regard to transition metal complexes of formula 9, the metal osmium is complexed to two substituted 2,2′-biimidazole ligands and one substituted or unsubstituted 2,2′-bipyridine ligand. R₁, R₂, R₃, R₄, R₅, R₆, R₁₆, R₁₇, R₁₈, R₁₉, R₂₀, R₂₁, R₂₂, R₂₃, c, d, and X are the same as described above.

In one embodiment, R₁ and R₂ are methyl; R₃, R₄, R₅, R₆, R₁₆, R₁₇, R₁₉, R₂₀, R₂₂ and R₂₃ are H; and R₁₈ and R₂₁ are the same and are H, methyl, or methoxy. In an embodiment of an embodiment of the invention, R₁₈ and R₂₁ are methyl or methoxy.

In another embodiment, R₁ and R₂ are methyl; R₃, R₄, R₅, R₆, R₁₆, R₁₇, R₁₈, R₁₉, R₂₀, R₂₂ and R₂₃ are H; and R₂₁ is halo, C1 to C12 alkoxy, C1 to C12 alkylamino, or C2 to C24 dialkylamino. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group. For example, R₂₁ is a C1 to C12 alkylamino or C2 to C24 dialkylamino, the alkyl portion(s) of which are substituted with a reactive group, such as a carboxylic acid, activated ester, or amine. Typically, the alkylamino group has 1 to 6 carbon atoms and the dialkylamino group has 2 to 8 carbon atoms.

With regard to transition metal complexes of formula 10, the metal osmium is complexed to two substituted 2,2′-biimidazole ligands and one substituted or unsubstituted 2-(2-pyridyl)imidazole ligand. R₁, R₂, R₃, R₄, R₅, R₆, R′₁, R′₃, R′₄, Rₐ, R_{b}, R_{c}, R_{d}, c, d, and X are the same as described above.

In one embodiment, R₁ and R₂ are methyl; R₃, R₄, R₅, R₆, R′₃, R′₄ and R_{d} are independently H or methyl; Rₐ and R_{c} are the same and are H; and R_{b} is C1 to C12 alkoxy, C1 to C12 alkylamino, or C2 to C24 dialkylamino. The alkyl or aryl portions of any of the substituents are optionally substituted by —F, —Cl, —Br, —I, alkylamino, dialkylamino, trialkylammonium (except on aryl portions), alkoxy, alkylthio, aryl, or a reactive group.

A list of specific examples of preferred transition metal complexes with respective redox potentials is shown in Table 1×.

TABLE 1
Redox Potentials of Selected Transition Metal Complexes

| | | E_{1/2} |
|---|---|---|
| | | (vs Ag/ |
| Complex | Structure | AgCl)/mV* |
| I | 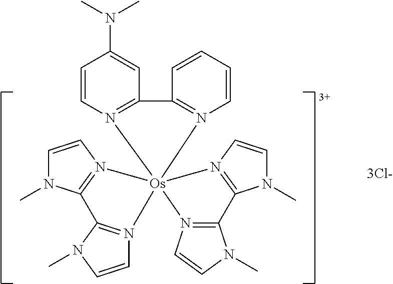 | −110 |
| | [Os(1,1′-dimethyl-2,2′-biimidazole)₂(4- | |
| | dimethylamino-2,2′-bipyridine)]Cl₃ | |
| II | 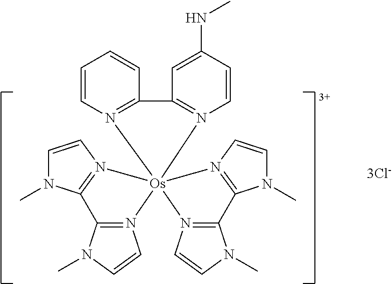 | −100 |
| | [Os(1,1′-dimethyl-2,2′-biimidazole)₂(4- | |
| | methylamino-2,2′-bipyridine)]Cl₃ | |
| III | 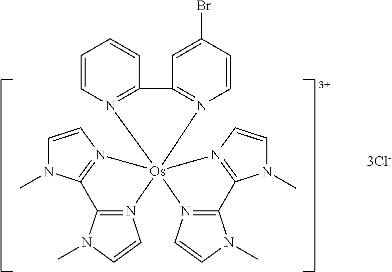 | 128 |
| | [Os(1,1′-dimethyl-2,2′-biimidazole)₂(4-bromo- | |
| | 2,2′-bipyridine)]Cl₃ | |
| IV | 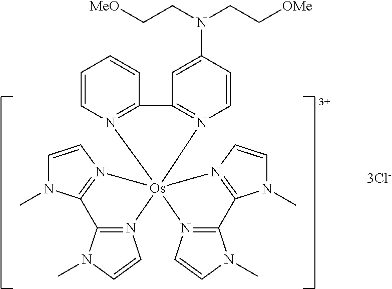 | −86 |
| | [Os(1,1′-dimethyl-2,2′-biimidazole)₂(4-di(2- | |
| | methoxyethyl)amino-2,2′-bipyridine)]Cl₃ | |
| V | 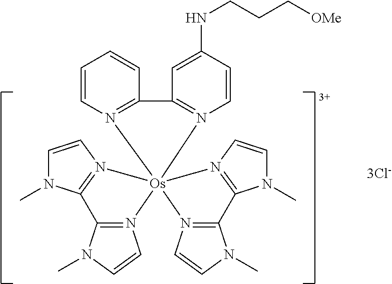 | −97 |
| | [Os(1,1′-dimethyl-2,2′-biimidazole)₂(4-(3- | |
| | methoxypropyl)amino-2,2′-bipyridine)]Cl₃ | |
| VI | 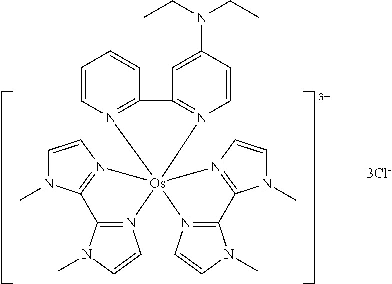 | −120 |
| | [Os(1,1′-dimethyl-2,2′-biimidazole)₂(4- | |
| | diethylamino-2,2′-bipyridine)]Cl₃ | |
| VII | 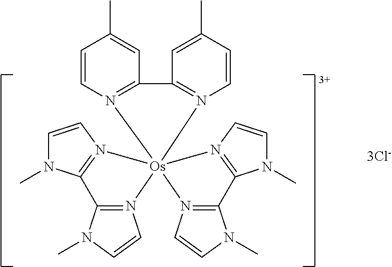 | 32 |
| | [Os(1,1′-dimethyl-2,2′-biimidazole)₂(4,4′- | |
| | dimethyl-2,2′-bipyridine)]Cl₃ | |
| VIII | 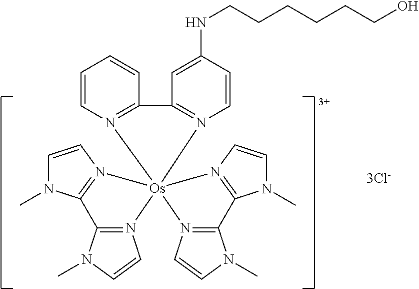 | −100 |
| | [Os(1,1′-dimethyl-2,2′-biimidazole)₂(4-(6- | |
| | hydroxyhexyl)amino-2,2′-bipyridine)]Cl₃ | |
| IX | 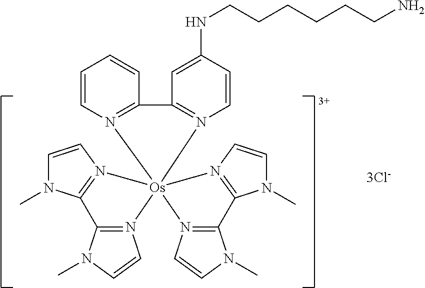 | −93 |
| | [Os(1,1′-dimethyl-2,2′-biimidazole)₂(4-(6- | |
| | aminohexyl)amino-2,2′-bipyridine)]Cl₃ | |
| X | 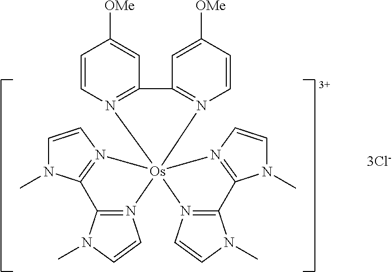 | −125 |
| | [Os(1,1′-dimethyl-2,2′-biimidazole)₂(4- | |
| | methoxypyridine)₂]Cl₃ | |
| XI | 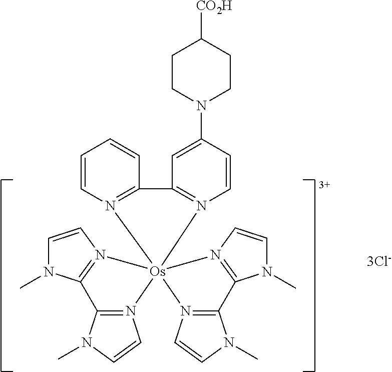 | −60 |
| | [Os(1,1′-dimethyl-2,2′-biimidazole)₂(4-(N-(4- | |
| | carboxy)piperidino)-2,2′-bipyridine)]Cl₃ | |
| XII | 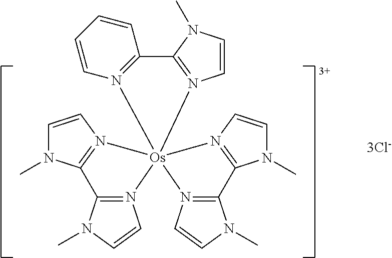 | −74 |
| | [Os(1,1′-dimethyl-2,2′-biimidazole)₂(1-methyl-2- | |
| | (2-pyridyl)imidazole)]Cl₃ | |
| XIII | 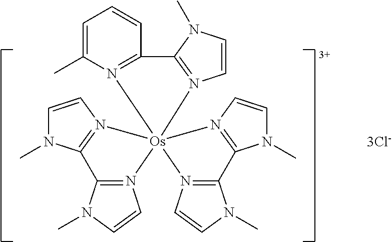 | −97 |
| | [Os(1,1′-dimethyl-2,2′-biimidazole)₂(1-methyl-2- | |
| | (6-methylpyrid-2-yl)imidazole)]Cl₃ | |
| IVX | 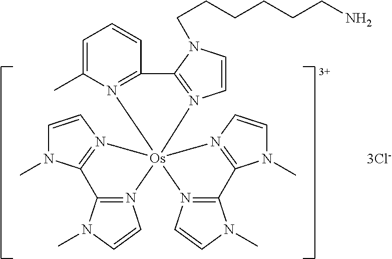 | −81 |
| | [Os(1,1′-dimethyl-2,2′-biimidazole)₂(1-(6- | |
| | aminohexyl)-2-(6-methylpyrid-2- | |
| | yl)imidazole)]Cl₃ | |
| VX | 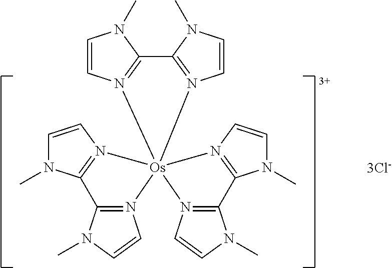 | −230 |
| | [Os(3,3′-dimethyl-2,2′-biimidazole)₂Cl₃ | |

*Redox potentials were estimated by averaging the positions of the reduction wave peaks and the oxidation wave peaks of cyclic voltammograms (CVs) obtained in pH 7 PBS buffer with a glassy carbon working electrode, a graphite counter electrode and a standard Ag/AgCl reference electrode at a sweep rate of 50 mV/s.

In some embodiments, the redox polymer comprises a transition metal complex, as described above, and a polymeric backbone. In some embodiments, the redox polymer comprises a transition metal complex (e.g. of Formula I) coupled to a polymeric backbone through one or more of L, L₁, L₂, L₃, and L₄. Additional examples of suitable transition metal complexes are described in U.S. Pat. No. 6,605,200, entitled “Polymeric Transition Metal Complexes and Uses Thereof”, incorporated herein by reference. In some embodiments, the polymeric backbone has functional groups that act as ligands of the transitional metal complex. Such polymeric backbones include, for example, poly(4-vinylpyridine) and poly(N-vinylimidazole) in which the pyridine and imidazole groups, respectively, can act as monodentate ligands of the transition metal complex. In some embodiments, the polymeric backbone comprises a poly(vinylpyridine)-based polymer.

In some embodiments, the polymeric backbone comprises a poly(vinylpyridine) having the following general formula, where n may be 2, n′ may be 17, and n″ may be 1:

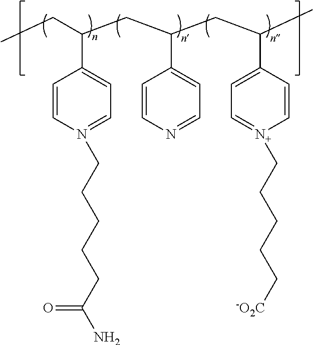

In other embodiments, the transition metal complex of the redox polymers of an embodiment of the invention can be the reaction product between a reactive group on a precursor polymer and a reactive group on a ligand of a precursor transition metal complex (such as a complex of Formula 1 where one of L, L₁, L₂, L₃ and L₄ includes a reactive group as described above). Suitable precursor polymers include, for example, poly(acrylic acid) (Formula 11), styrene/maleic anhydride copolymer (Formula 12), methylvinylether/maleic anhydride copolymer (GANTREX polymer) (Formula 13), poly(vinylbenzylchloride) (Formula 14), poly(allylamine) (Formula 15), polylysine (Formula 16), carboxy-poly(vinylpyridine (Formula 17), and poly(sodium 4-styrene sulfonate) (Formula 18).

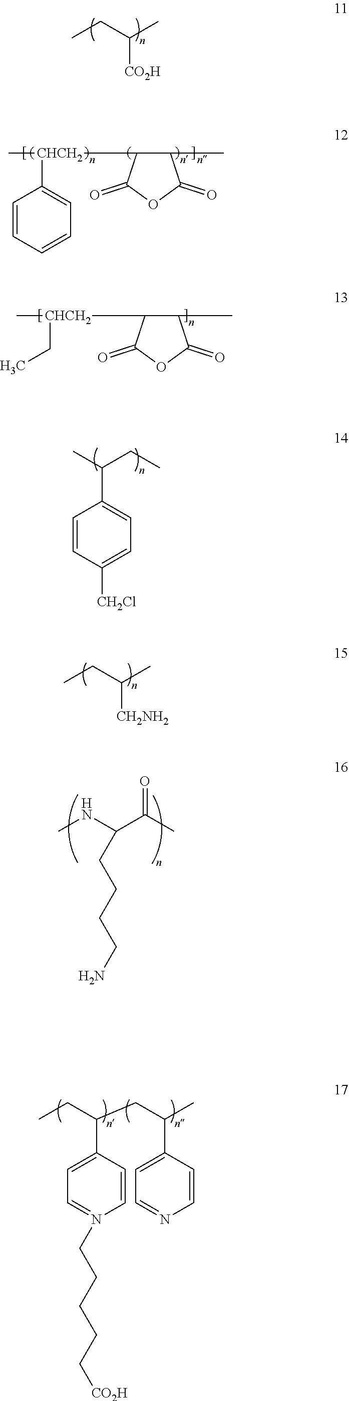

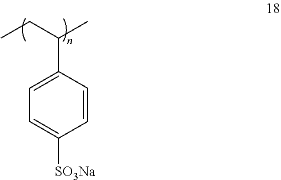

Alternatively, the transition metal complex can have reactive group(s) for immobilization or conjugation of the complexes to other substrates or carriers, examples of which include, but are not limited to, macromolecules (e.g. enzymes) and surfaces (e.g. electrode surfaces). For reactive attachment to polymers, substrates, or other carriers, the transition metal complex precursor includes at least one reactive group that reacts with a reactive group on the polymer, substrate, or carrier. Typically, covalent bonds are formed between the two reactive groups to generate a linkage. Examples of such linkages are provided in Table 2, below. Generally, one of the reactive groups is an electrophile and the other reactive group is a nucleophile.

TABLE 2
Examples of Reactive Group Linkages

| First | Second | Resulting |
|---|---|---|
| Reactive Group | Reactive Group | Linkage |
| Activated ester* | Amine | Carboxamide |
| Acrylamide | Thiol | Thioether |
| Acyl azide | Amine | Carboxamide |
| Acyl halide | Amine | Carboxamide |
| Carboxylic acid | Amine | Carboxamide |
| Aldehyde or ketone | Hydrazine | Hydrazone |
| Aldehyde or ketone | Hydroxyamine | Oxime |
| Alkyl halide | Amine | Alkylamine |
| Alkyl halide | Carboxylic acid | Carboxylic ester |
| Alkyl halide | Imidazole | Imidazolium |
| Alkyl halide | Pyridine | Pyridinium |
| Alkyl halide | Alcohol/phenol | Ether |
| Alkyl halide | Thiol | Thioether |
| Alkyl sulfonate | Thiol | Thioether |
| Alkyl sulfonate | Pyridine | Pyridinium |
| Alkyl sulfonate | Imidazole | Imidazolium |
| Alkyl sulfonate | Alcohol/phenol | Ether |
| Anhydride | Alcohol/phenol | Ester |
| Anhydride | Amine | Carboxamide |
| Aziridine | Thiol | Thioether |
| Aziridine | Amine | Alkylamine |
| Aziridine | Pyridine | Pyridinium |
| Epoxide | Thiol | Thioether |
| Epoxide | Amine | Alkylamine |
| Epoxide | Pyridine | Pyridinium |
| Halotriazine | Amine | Aminotriazine |
| Halotriazine | Alcohol | Triazinyl ether |
| Imido ester | Amine | Amidine |
| Isocyanate | Amine | Urea |
| Isocyanate | Alcohol | Urethane |
| Isothiocyanate | Amine | Thiourea |
| Maleimide | Thiol | Thioether |
| Sulfonyl halide | Amine | Sulfonamide |

*Activated esters, as understood in the art, generally include esters of succinimidyl, benzotriazolyl, or aryl substituted by electron-withdrawing groups such as sulfo, nitro, cyano, or halo; or carboxylic acids activated by carbodiimides.

Transition metal complexes used in the redox polymers of embodiments of an embodiment of the invention can be soluble in water or other aqueous solutions, or in organic solvents. In general, transition metal complexes can be made soluble in either aqueous or organic solvents by having an appropriate counter ion or ions, X. For example, transition metal complexes with small counter anions, such as F⁻, Cl⁻, and Br⁻, tend to be water soluble. On the other hand, transition metal complexes with bulky counter anions, such as I⁻, BF₄⁻ and PF₆⁻, tend to be soluble in organic solvents. In an embodiment of an embodiment of the invention, the solubility of transition metal complexes of aspects of an embodiment of the invention is greater than about 0.1 M (moles/liter) at 25° C. for a desired solvent.

The use of transition metal complexes as redox mediators is described, for example, in U.S. Pat. Nos. 5,262,035; 5,262,305; 5,320,725; 5,365,786; 5,593,852; 5,665,222; 5,972,199; 6,143,164; 6,134,461; 6,175,752; 6,338,790; and 6,616,819, all of which are herein incorporated by reference. The transitional metal complexes described herein can typically be used in place of those discussed in the references listed above.

Redox polymers of the invention may comprise, inter alia, a transition metal complex, a crosslinker and a polymeric backbone. Such redox polymers are useful as redox mediators in electrochemical biosensors for the detection of analytes in bio-fluids, for example. In general, the redox polymer is disposed on or in proximity to (e.g. in a solution surrounding) a working electrode. The redox polymer transfers electrons between the working electrode and an analyte. In some preferred embodiments, an enzyme is also included to facilitate the transfer. For example, the redox polymer transfers electrons between the working electrode and glucose (typically via an enzyme) in an enzyme-catalyzed reaction of glucose. Redox polymers are particularly useful for forming non-leachable coatings on the working electrode. These can be formed, for example, by crosslinking the redox polymer on the working electrode, or by cross-linking the redox polymer and the enzyme on the working electrode.

Transition metal complexes can enable accurate, reproducible and quick or continuous assays. Transition metal complex redox mediators accept electrons from, or transfer electrons to, enzymes or analytes at a high rate and also exchange electrons rapidly with an electrode. Typically, the rate of self exchange, the process in which a reduced redox mediator transfers an electron to an oxidized redox mediator, is rapid. At a defined redox mediator concentration, this provides for more rapid transport of electrons between the enzyme (or analyte) and electrode, and thereby shortens the response time of the sensor. Additionally, the transition metal complex redox mediators disclosed herein are typically stable under ambient light and at the temperatures encountered in use, storage and transportation. Preferably, the transition metal complex redox mediators do not undergo chemical change, other than oxidation and reduction, in the period of use or under the conditions of storage, though the redox mediators can be designed to be activated by reacting, for example, with water or the analyte.

A transition metal complex can be used as a redox mediator in combination with a redox enzyme to electrooxidize or electroreduce the analyte or a compound derived of the analyte, for example by hydrolysis of the analyte. The redox potentials of the redox mediators are generally more positive (i.e. more oxidizing) than the redox potentials of the redox enzymes when the analyte is electrooxidized and more negative when the analyte is electroreduced. For example, the redox potentials of the preferred transition metal complex redox mediators used for electrooxidizing glucose with glucose oxidase or PQQ-glucose dehydrogenase as enzyme is between about −200 mV and +200 mV versus a Ag/AgCl reference electrode, and the most preferred mediators have redox potentials between about −100 mV and about +100 mV versus a Ag/AgCl reference electrode.

Electron transport involves an exchange of electrons between segments of the redox polymers (e.g. one or more transition metal complexes coupled to a polymeric backbone, as described above) in a crosslinked film disposed on an electrode. A transition metal complex can be bound to the polymer backbone though covalent, coordinative or ionic bonds, where covalent and coordinative binding are preferred. Electron exchange occurs, for example, through the collision of different segments of the crosslinked redox polymer. Electrons transported through the redox polymer can originate from, for example, electrooxidation or electroreduction of an enzymatic substrate, such as, for example, the oxidation of glucose by glucose oxidase.

The degree of crosslinking of the redox polymer can influence the transport of electrons or ions and thereby the rates of the electrochemical reactions. Excessive crosslinking of the polymer can reduce the mobility of the segments of the redox polymer. A reduction in segment mobility can slow the diffusion of electrons or ions through the redox polymer film. A reduction in the diffusivity of electrons, for example, can require a concomitant reduction in the thickness of the film on the electrode where electrons or electron vacancies are collected or delivered. The degree of crosslinking in a redox polymer film can thus affect the transport of electrons from, for example, an enzyme to the transition metal redox centers of the redox polymer such as, for example, Os^{2+/3+} metal redox centers; between redox centers of the redox polymer; and from these transition metal redox centers to the electrode.

Inadequate crosslinking of a redox polymer can result in excessive swelling of the redox polymer film and to the leaching of the components of the redox polymer film. Excessive swelling can also result in the migration of the swollen polymer into the analyzed solution, in the softening of the redox polymer film, in the film's susceptibility to removal by shear, or any combination of these effects.

Crosslinking can decrease the leaching of film components and can improve the mechanical stability of the film under shear stress. For example, as disclosed in Binyamin, G. and Heller, A; Stabilization of Wired Glucose Oxidase Anodes Rotating at 1000 rpm at 37° C.; Journal of the Electrochemical Society, 146(8), 2965-2967, 1999, herein incorporated by reference, replacing a difunctional crosslinker, such as polyethylene glycol diglycidyl ether, with a trifunctional crosslinker such as N,N-diglycidyl-4-glycidyloxyaniline, for example, can reduce leaching and shear problems associated with inadequate crosslinking.

In some embodiments, the sensing layer comprises a redox polymer comprising polymeric backbone, a crosslinker, and a transition metal complex. Suitable crosslinking agents (e.g. crosslinkers) may include, but are not limited to, polyethylene glycol diglycidylether (PEGDGE), glycerol triglycidyl ether (Gly3), polydimethylsiloxane diglycidylether (PDMS-DGE), or other polyepoxides, cyanuric chloride, N-hydroxysuccinimide, imidoesters, epichlorohydrin, or derivatized variants thereof, and any combination thereof. In some embodiments, the crosslinker is an epoxide. In some embodiments, the epoxide is a triglycidyl ether. Branched versions with similar terminal chemistry are also suitable for the present disclosure. For example, in some embodiments, the crosslinking agent can be glycerol triglycidyl ether and/or PEGDGE and/or polydimethylsiloxane diglycidylether (PDMS-DGE). In some embodiments, the crosslinker is a bifunctional crosslinker, a trifunctional crosslinker, or a tetrafunctional crosslinker. Examples of other bifunctional, trifunctional and tetrafunctional crosslinkers are listed below:

### Amine-Reactive Bifunctional Crosslinkers

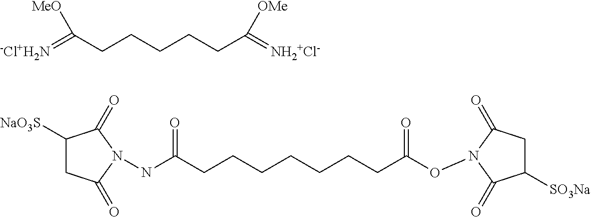

### Pyridine- or Imidazole-Reactive Bifunctional Crosslinkers

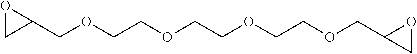 [SMILES: C(COCCOCC1CO1)OCCOCC1CO1]

### Pyridine- or Imidazole-Reactive Trifunctional Crosslinker

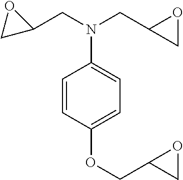 [SMILES: c1cc(N(CC2CO2)CC2CO2)ccc1OCC1CO1]

### Pyridine- or Imidazole-Reactive Tetrafunctional Crosslinkers

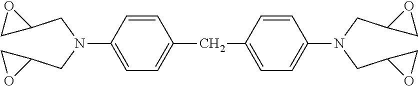 [SMILES: c1cc(N(CC2CO2)CC2CO2)ccc1Cc1ccc(N(CC2CO2)CC2CO2)cc1]

Alternatively, the number of crosslinking sites can be increased by reducing the number of transition metal complexes attached to the polymeric backbone, thus making more polymer pendant groups available for crosslinking. One important advantage of at least some of the redox polymers is the increased mobility of the pendant transition metal complexes, resulting from the flexibility of the pendant groups. As a result, in at least some embodiments, fewer transition metal complexes per polymer backbone are needed to achieve a desired level of diffusivity of electrons and current density of analyte electrooxidation or electroreduction.

Transition metal complexes can be directly or indirectly attached to a polymeric backbone, depending on the availability and nature of the reactive groups on the complex and the polymeric backbone. For example, the pyridine groups in poly(4-vinylpyridine) or the imidazole groups in poly(N-vinylimidazole) are capable of acting as monodentate ligands and thus can be attached to a metal center directly. Alternatively, the pyridine groups in poly(4-vinylpyridine) or the imidazole groups in poly(N-vinylimidazole) can be quaternized with a substituted alkyl moiety having a suitable reactive group, such as a carboxylate function, that can be activated to form a covalent bond with a reactive group, such as an amine, of the transition metal complex. (See Table 2 for a list of other examples of reactive groups).

Redox centers such as, for example, Os^{2+/3+} can be coordinated with five heterocyclic nitrogens and an additional ligand such as, for example, a chloride anion. An example of such a coordination complex includes two bipyridine ligands which form stable coordinative bonds, the pyridine of poly(4-vinylpyridine) which forms a weaker coordinative bond, and a chloride anion which forms the least stable coordinative bond.

Alternatively, redox centers, such as Os^{2+/3+}, can be coordinated with six heterocyclic nitrogen atoms in its inner coordination sphere. The six coordinating atoms are preferably paired in the ligands, for example, each ligand is composed of at least two rings. Pairing of the coordinating atoms can influence the potential of an electrode used in conjunction with redox polymers of the present invention.

Typically, for analysis of glucose, the potential at which the working electrode, coated with the redox polymer, is poised is negative of about +250 mV vs. SCE (standard calomel electrode). Preferably, the electrode is poised negative of about +150 mV vs. SCE. Poising the electrode at these potentials reduces the interfering electrooxidation of constituents of biological solutions such as, for example, urate, ascorbate and acetaminophen. The potential can be modified by altering the ligand structure of the complex.

The redox potential of a redox polymer, as described herein, is related to the potential at which the electrode is poised. Selection of a redox polymer with a desired redox potential allows tuning of the potential at which the electrode is best poised. The redox potentials of a number of the redox polymers described herein are negative of about +150 mV vs. SCE and can be negative of about +50 mV vs. SCE to allow the poising of the electrode potentials negative of about +250 mV vs. SCE and preferably negative of about +150 mV vs. SCE.

The strength of the coordination bond can influence the potential of the redox centers in the redox polymers. Typically, the stronger the coordinative bond, the more positive the redox potential. A shift in the potential of a redox center resulting from a change in the coordination sphere of the transition metal can produce a labile transition metal complex. For example, when the redox potential of an Os^{2+/3+} complex is downshifted by changing the coordination sphere, the complex becomes labile. Such a labile transition metal complex may be undesirable when fashioning a metal complex polymer for use as a redox mediator and can be avoided through the use of weakly coordinating multidentate or chelating heterocyclics as ligands.

Transition metal complexes used as redox mediators in electrodes can be affected by the presence of transition metals in the analyzed sample including, for example, Fe³⁺ or Zn²⁺. The addition of a transition metal cation to a buffer used to test an electrode results in a decline in the current produced. The degree of current decline depends on the presence of anions in the buffer which precipitate the transition metal cations. The lesser the residual concentration of transition metal cations in the sample solution, the more stable the current. Anions which aid in the precipitation of transition metal cations include, for example, phosphate. It has been found that a decline in current upon the addition of transition metal cations is most pronounced in non-phosphate buffers. If an electrode is transferred from a buffer containing a transition metal cation to a buffer substantially free of the transition metal cation, the original current is restored.

The decline in current is thought to be due to additional crosslinking of a pyridine-containing polymer backbone produced by the transition metal cations. The transition metal cations can coordinate nitrogen atoms of different chains and chain segments of the polymers. Coordinative crosslinking of nitrogen atoms of different chain segments by transition metal cations can reduce the diffusivity of electrons.

Serum and other physiological fluids contain traces of transition metal ions, which can diffuse into the films of electrodes made with the redox polymers of the present invention, lowering the diffusivity of electrons and thereby the highest current reached at high analyte concentration. In addition, transition metal ions like iron and copper can bind to proteins of enzymes and to the reaction centers or channels of enzymes, reducing their turnover rate. The resulting decrease in sensitivity can be remedied through the use of anions which complex with interfering transition metal ions, for example, in a buffer employed during the production of the transition metal complex. A non-cyclic polyphosphate such as, for example, pyrophosphate or triphosphate, can be used. For example, sodium or potassium non-cyclic polyphosphate buffers can be used to exchange phosphate anions for those anions in the transition metal complex which do not precipitate transition metal ions. The use of linear phosphates can alleviate the decrease in sensitivity by forming strong complexes with the damaging transition metal ions, assuring that their activity will be low. Other complexing agents can also be used as long as they are not electrooxidized or electroreduced at the potential at which the electrode is poised.

Glucose oxidase is a flavoprotein enzyme that catalyzes the oxidation by dioxygen of D-glucose to D-glucono-1,5-lactone and hydrogen peroxide. Reduced transition metal cations such as, for example, Fe²⁺, and some transition metal complexes, can react with hydrogen peroxide. These reactions form destructive OH radicals and the corresponding oxidized cations. The presence of these newly formed transition metal cations can inhibit the enzyme and react with the metal complex. Also, the oxidized transition metal cation can be reduced by the FADH₂ centers of an enzyme, or by the transition metal complex.

Inhibition of the active site of an enzyme or a transition metal complex by a transition metal cation, as well as damaging reactions with OH radicals can be alleviated, thus increasing the sensitivity and functionality of the electrodes by incorporating non-cyclic polyphosphates, as discussed above. Because the polyphosphate/metal cation complex typically has a high (oxidizing) redox potential, its rate of oxidation by hydrogen peroxide is usually slow. Alternatively, an enzyme such as, for example, catalase, can be employed to degrade hydrogen peroxide.

### Membrane Polymer

In some embodiments, the device comprises a membrane polymer (also referred to herein as the polymeric membrane). In some embodiments, the membrane polymer is a mass transport limiting membrane. In some embodiments, the membrane polymer comprises a crosslinked polyvinylpyridine homopolymer or copolymer. The composition of the membrane polymer may be the same or different when the membrane coats multiple active areas of differing types. When the membrane composition varies at two different locations, the membrane may comprise a bilayer membrane or a homogeneous admixture of two different membrane polymers, one of which may be a crosslinked polyvinylpyridine or polyvinylimidazole homopolymer or copolymer. Suitable techniques for depositing a membrane polymer upon the active area may include, for example, spray coating, painting, inkjet printing, screen printing, stenciling, roller coating, dip coating, the like, and any combination thereof. Dip coating techniques may be especially desirable for polyvinylpyridine and polyvinylimidazole polymers and copolymers.

In certain embodiments, the membrane polymer is composed of crosslinked polymers containing heterocyclic nitrogen groups, such as polymers of polyvinylpyridine and polyvinylimidazole. In some embodiments, a membrane may be formed by crosslinking in situ a polymer, including those discussed above, modified with a zwitterionic moiety, a non-pyridine copolymer component, and optionally another moiety that is either hydrophilic or hydrophobic, and/or has other desirable properties, in a buffer solution (e.g. an alcohol-buffer solution). The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, and the like, and any combinations thereof, may be used to enhance the biocompatibility of the polymer or the resulting membrane.

In some embodiments, the membrane polymer may comprise a compound including, but not limited to, poly(styrene-co-maleic anhydride), dodecylamine and poly(propylene glycol)-block-poly(ethylene glycol)-block-poly(propylene glycol) (2-aminopropyl ether) crosslinked with poly(propylene glycol)-block-poly(ethylene glycol)-block-poly(propylene glycol) bis(2-aminopropyl ether); poly(N-isopropyl acrylamide); a copolymer of poly(ethylene oxide) and poly(propylene oxide); polyvinylpyridine; a derivative of polyvinylpyridine; polyvinylimidazole; a derivative of polyvinylimidazole; polyvinylpyrrolidone (PVP), and the like; and any combination thereof. In some embodiments, the membrane polymer may be comprised of a polyvinylpyridine-co-styrene polymer. In some embodiments, the membrane polymer is comprised of a polyvinylpyridine-co-styrene polymer in which a portion of the pyridine nitrogen atoms are functionalized with a non-crosslinked poly(ethylene glycol) tail and a portion of the pyridine nitrogen atoms are functionalized with an alkylsulfonic acid group. Other membrane compounds, alone or in combination with any aforementioned membrane compounds, may comprise a suitable copolymer of 4-vinylpyridine and styrene and an amine-free polyether arm.

The membrane polymers described herein may further be crosslinked with one or more crosslinking agents, including those listed above with reference to the enzyme described herein and with reference to the redox polymers described herein. For example, suitable crosslinking agents (e.g. crosslinkers) may include, but are not limited to, polyethylene glycol diglycidylether (PEGDGE), glycerol triglycidyl ether (Gly3), polydimethylsiloxane diglycidylether (PDMS-DGE), or other polyepoxides, cyanuric chloride, N-hydroxysuccinimide, imidoesters, epichlorohydrin, or derivatized variants thereof, and any combination thereof. In some embodiments, the crosslinker is an epoxide. In some embodiments, the epoxide is a triglycidyl ether. Branched versions with similar terminal chemistry are also suitable for the present disclosure. For example, in some embodiments, the crosslinking agent can be glycerol triglycidyl ether and/or PEGDGE and/or polydimethylsiloxane diglycidylether (PDMS-DGE). In some embodiments, a weight ratio of the polymer to the crosslinker is from about 4:1 to about 32:1 For example, in some embodiments the weight ratio of the polymer to the crosslinker is from about 8:1 to about 16:1. In some embodiments, the polymeric membrane further comprises a layer of poly(ethylene glycol).

A membrane polymer may be formed in situ by applying an alcohol-buffer solution of a crosslinker and a modified polymer over the active area and any additional compounds included in the active area (e.g. electron transfer agent) and allowing the solution to cure for about one to two days or other appropriate time period. The crosslinker-polymer solution may be applied over the active area by placing a droplet or droplets of the membrane solution on at least the sensor element(s) of the sensor tail, by dipping the sensor tail into the membrane solution, by spraying the membrane solution on the sensor, by heat pressing or melting the membrane in any sized layer (such as discrete or all encompassing) and either before or after singulation, vapor deposition of the membrane, powder coating of the membrane, and the like, and any combination thereof. In order to coat the distal and side edges of the sensor, the membrane material may be applied subsequent to application (e.g. singulation) of the sensor electronic precursors (e.g. electrodes). In some embodiments, the analyte sensor is dip-coated following electronic precursor application to apply one or more membranes. Alternatively, the analyte sensor could be slot-die coated wherein each side of the analyte sensor is coated separately. A membrane applied in the above manner may have any of various functions including, but not limited to, mass transport limitation (i.e. reduction or elimination of the flux of one or more analytes and/or compounds that reach the active area), biocompatibility enhancement, interferent reduction, and the like, and any combination thereof.

Generally, the thickness of the membrane polymer is controlled by the concentration of the membrane solution, by the number of droplets of the membrane solution applied, by the number of times the sensor is dipped in the membrane solution, by the volume of membrane solution sprayed on the sensor, and the like, and by any combination of these factors. In some embodiments, the membrane polymer may have a thickness ranging from about 0.1 micrometers (μm) to about 1000 μm, encompassing any value and subset therebetween. As stated above, the membrane polymer may overlay one or more active areas, and in some embodiments, the active areas may have a thickness of from about 0.1 μm to about 50 μm, encompassing any value and subset therebetween. In some embodiments, a series of droplets may be applied atop one another to achieve the desired thickness of the active area and/or membrane, without substantially increasing the diameter of the applied droplets (i.e. maintaining the desired diameter or range thereof). Each single droplet, for example, may be applied and then allowed to cool or dry, followed by one or more additional droplets. Active areas and membrane may, but need not be, the same thickness throughout or composition throughout.

In some embodiments, the membrane polymer comprises polydimethylsiloxane (PDMS), polydimethylsiloxane diglycidylether (PDMS-DGE), aminopropyl terminated polydimethylsiloxane, and the like, and any combination thereof for use as a leveling agent (e.g. for reducing the contact angle of the membrane composition or active area composition). Branched versions with similar terminal chemistry are also suitable for the present disclosure. Certain leveling agents may additionally be included, such as those found, for example, in U.S. Pat. No. 8,983,568, the disclosure of which is incorporated by reference herein in its entirety.

In some instances, the membrane polymer may form one or more bonds with the active area. As used herein, the term “bonds,” and grammatical variants thereof, refers to any type of an interaction between atoms or molecules that allows chemical compounds to form associations with each other, such as, but not limited to, covalent bonds, ionic bonds, dipole-dipole interactions, hydrogen bonds, London dispersion forces, and the like, and any combination thereof. For example, in situ polymerization of the membrane can form crosslinks between the polymers of the membrane and the polymers in the active area. In some embodiments, crosslinking of the membrane to the active area facilitates a reduction in the occurrence of delamination of the membrane from the sensor.

In some embodiments, the membrane polymer comprises a poly(vinylpyridine-co-styrene) copolymer. In some embodiments, the membrane polymer comprises:

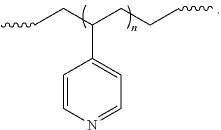

In some embodiments, the membrane polymer is derivatized by the addition of propylsulfonate and poly(ethyleneoxide) moieties. In some embodiments, the membrane polymer comprises.

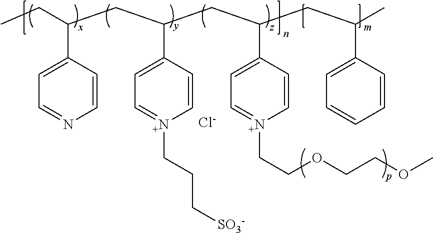

In some embodiments, x=0.85, y=0.1, z=0.05, n=9, m=1, and p=about 10. This formulation is referred to as 10Q5.

In some embodiments, the membrane polymer comprises a poly(vinylpyridine-co-styrene) copolymer of high molecular weight, that is cross-linked using a trifunctional, short-chain epoxide, such as glycerol triglycidyl ether. In some embodiments, the polymeric membrane is about 50 m thick. In some embodiments, the polymeric membrane serves to reduce glucose diffusion to the active sensing layer by a factor of about 50. The polymeric membrane also provides a surface that is biocompatible, such that bodily irritation from the subcutaneous portion of the sensor is reduced.

In some embodiments, the membrane polymer preparation may comprise 16 mg/mL of 10Q5, as depicted above (wherein x=0.85, y=0.1, z=0.05, n=9, m=1, and p=about 10), 8 mg/ml glycerol triglycidyl ether (the cross-linker), and optionally 7.5 mg/ml manganese 5,10,15,20-tetra(4-pyridyl)-21H 23H-porphine chloride (MnTPyP), a compound possessing both superoxide dismutase and catalase activity. 10Q5 is based on a poly(vinylpyridine-co-styrene) copolymer, which has been further derivitized by the addition of propylsulfonate and poly(ethyleneoxide) moieties.

In some embodiments, the membrane polymer comprises a polymer described in U.S. Pat. No. 6,932,894, incorporated herein by reference in its entirety. In some embodiments, the membrane polymer comprises a heterocyclic nitrogen containing polymer. In some embodiments, the membrane polymer comprises the formula:

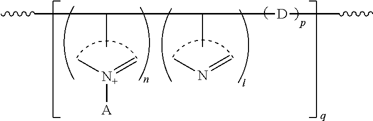

wherein the solid horizontal line represents a polymer backbone;
A is an alkyl substituted with a water-soluble constituent;
D is selected from a group consisting of phenylalkyl, alkoxystyrene, hydroxyalkyl, alkoxyalkyl, alkoxycarbonylalkyl, a poly(ethylene glycol)-containing constituent, and a polyhydroxyl-containing constituent; and each of n, l, p and q is independently a positive number.

In some embodiments, A is positively charged. In some embodiments, A is selected from a group consisting of a sulfonate, a carboxylate, and a phosphate. In some embodiments, A is selected from a group consisting of sulfopropyl, sulfobutyl, carboxypropyl, and carboxypentyl. In some embodiments, A is of the formula L-G, where L is a C2-C12 linear or branched alkyl linker and G is a negatively charged carboxy or sulfonate. L may be substituted with an aryl, alkoxy, alkenyl, alkynyl, —F, —Cl, —OH, aldehyde, ketone, ester, or amide. In some embodiments, D is styrene or C1-C18 alkyl methacrylate.

Exemplary membrane polymers are set forth below:

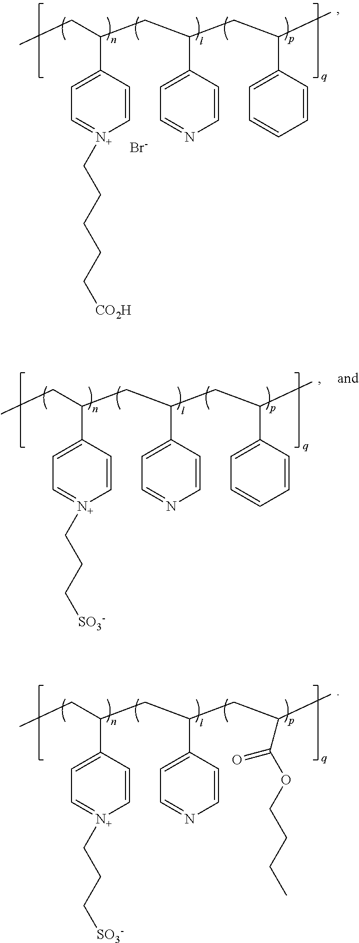

In some embodiments, the membrane polymer further comprises a B-containing copolymer such that the polymer has the formula:

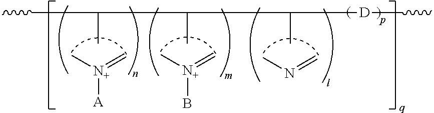

wherein B is a modifier and m is a positive number. In some embodiments, B is selected from a group consisting of a chelator, a negatively charged constituent, a hydrophobic hydrocarbon constituent, a hydrophilic hydroxyl or polyhydroxy constituent, a silicon polymer, and a poly(ethylene glycol). For example, B may be a poly(ethylene glycol) having a molecular weight of from about 100 to about 20,000.

In some embodiments, the membrane polymer has a formula selected from a group consisting of:

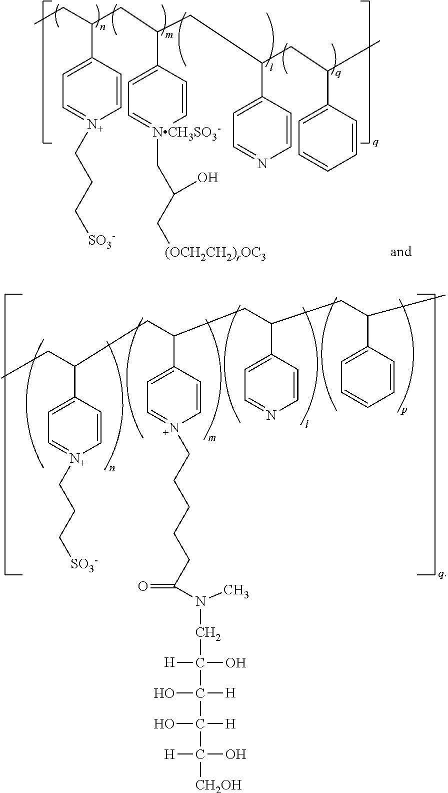

For the polymeric membrane structures shown above, D is a component of a poly(heterocyclic nitrogen-co-D) polymer. Examples of D include, but are not limited to, phenylalkyl, alkoxystyrene, hydroxyalkyl, alkoxyalkyl, alkoxycarbonylalkyl, and a molecule containing a poly(ethylene glycol) or polyhydroxyl group. Some poly(heterocyclic nitrogen-co-D) polymers suitable as starting materials for the present invention are commercially available. For example, poly(2-vinylpyridine-co-styrene), poly(4-vinylpyridine-co-styrene) and poly(4-vinylpyridine-co-butyl methacrylate) are available from Aldrich Chemical Company, Inc. Other poly(heterocyclic nitrogen-co-D) polymers can be readily synthesized by anyone skilled in the art of polymer chemistry using well-known methods. Preferably, D is a styrene or a C1-C18 alkyl methacrylate component of a polyvinylpyridine-poly-D, such as (4-vinylpyrine-co-styrene) or poly(4-vinylpyridine-co-butyl methacrylate), more preferably, the former. D may contribute to various desirable properties of the membrane including, but not limited to, hydrophobicity, hydrophilicity, solubility, biocompatibility, elasticity and strength. D may be selected to optimize or “fine-tune” a membrane made from the polymer in terms of its permeability to an analyte and its non-permeability to an undesirable, interfering component, for example. For the polymeric membrane structures shown above, m is an average number of an associated polymer unit or polymer units shown in the closest parentheses to the left, and the letters n, l, and p designate, respectively, an average number of each copolymer component in each polymer unit. The letter q is one for a block copolymer or a number greater than one for a copolymer with a number of repeating polymer units. By way of example, the q value for a polymer of the present invention may be ≥about 950, where n, l and p are 1, 8 and 1, respectively. The letter q is thus related to the overall molecular weight of the polymer. Preferably, the average molecular weight of the polymer is above about 50,000, more preferably above about 200,000, most preferably above about 1,000,000.

In some embodiments, at least one heterocyclic nitrogen constituent of the polymer is independently selected from a group consisting of pyridine, imidazole, oxazole, thiazole, pyrazole, and any derivative thereof. In some embodiments, at least one heterocyclic nitrogen constituent of the polymer is independently selected from a group consisting of 2-vinylpyridine, 3-vinylpyridine, 4-vinylpyridine, 1-vinylimidazole, 2-vinylimidazole, and 4-vinylimidazole.

Such polymeric membranes may be employed in a variety of sensors, such as the two- or three-electrode sensors described previously in detail in U.S. Pat. No. 6,932,894 of Mao et al., published on Mar. 6, 2003, which is incorporated in its entirety herein by this reference. By way of example, the membrane may be used in a two-electrode amperometric glucose sensor, as shown in FIG. 4. The amperometric glucose sensor of FIG. 4 comprises a substrate disposed between a working electrode that is typically carbon-based, and an Ag/AgCl counter/reference electrode. A sensing layer is disposed on the working electrode. A polymeric membrane encapsulates the entire glucose sensor, including the Ag/AgCl counter/reference electrode. The sensing layer of the glucose sensor consists of crosslinked glucose oxidase and redox polymer. The redox polymer comprises a low potential polymeric osmium complex mediator, as disclosed in Published PCT Application, International Publication No. WO 01/36660 A2, the entire contents of which are incorporated herein by reference. The enzyme- and mediator-containing formulation that can be used in the sensing layer, and methods for applying them to an electrode system, are known in the art, for example, from the above-mentioned U.S. Pat. No. 6,134,461 of Say et al.

In some embodiments, the sensing layer is coated with the polymeric membrane by a dipping process. In some embodiments, the dipping process comprises dipping the sensing layer into a membrane solution comprising the polymeric membrane material and the crosslinker. For example, in some embodiments the sensing layer is coated with the polymeric membrane by a dipping process involving dipping the sensing layer into a membrane solution comprising the poly(vinylpyridine-co-styrene copolymer (e.g. the polymeric membrane material) and the cross linker (e.g. PEGDGE, Gly3). In some embodiments, the sensing layer is dipped multiple times into the membrane solution. In some embodiments, the sensing layer is dipped twice into the membrane solution. In some embodiments, the sensing layer is dipped three times into the membrane solution. In some embodiments, the sensor is cured after dipping. In some embodiments, the sensor is cured at ambient temperature and normal humidity for at least 24 hours (e.g. 1 day, 2 days, 3 days, etc.).

### Carbohydrates

In some embodiments, the sensing layer and/or the membrane polymer comprise a carbohydrate. In some embodiments, the analyte sensor comprising a carbohydrate displays increased stability and performance compared to an equivalent analyte sensor lacking the carbohydrate in the sensing layer and/or membrane polymer. In some embodiments, the sensing layer comprises glucose oxidase enzyme.

In some embodiments, the carbohydrate is a pyranose, furanose, disaccharide, oligosaccharide, polysaccharide, iminosugar, sugar alcohol, glycoside, amino sugar, or a combination thereof. The term “pyranose” refers to a monosaccharide that forms a six-membered ring. In some embodiments, the pyranose is glucose, galactose, mannose, or a combination thereof. The term “furanose” refers to a monosaccharide that forms a five-membered ring. In some embodiments, the furanose is fructose, xylose, ribose, or a combination thereof. The term “disaccharide” refers to a combination of 2 pyranose and/or furanose units (e.g. two pyranose units, two furanose units, or one pyranose and one furanose unit). In some embodiments, the disaccharide is lactose, maltose, cellobiose, or a combination thereof. The term “oligosaccharide” typically refers to a saccharide containing 3-10 monosaccharide units, whereas the term “polysaccharide” typically refers to a saccharide containing ore than 10 monosaccharide units. In some embodiments, the oligo- or polysaccharide is starch, maltotriose, amylose, cellulose, inulin, or a combination thereof. In some embodiments, the oxygen present in a 5 or 6 membered ring can be substituted with a suitable moiety such as carbon, nitrogen, or sulfur. For example, the carbohydrate may be the iminosugar nojirimycin. In some embodiments, the carbohydrate comprises a 5 or 6 membered ring with 3 or more hydroxyl groups and any number of additional carbon atoms attached to the ring. For example, the carbohydrate may be the sugar alcohol inositol.

In some embodiments, the iminosugar is nojirimycin. In some embodiments, the sugar alcohol is inositol. In some embodiments, the glycoside is a glucoside. In some embodiments, the glucoside is 1-O-methyl glucose. In some embodiments, the glycoside comprises an aryl group. In some embodiments, the glycoside comprises an acyl group. In some embodiments, the amino sugar is a glucosamine or N-acetylglucosamine.

In some embodiments, the carbohydrate comprises hydroxyls substituted to form O—R groups, with R including alkyl, aryl, acyl. Exemplary substituted carbohydrates include, for example, 1-O-methyl glucose, pentaacetylglucose, etc. In some embodiments, the carbohydrate comprises an exo-ring oxygen that is substituted with nitrogen, as in glucosamine or N-acetylglucosamine.

In some embodiments, gluconolactone is a stabilizing compound in the sensors provided herein. In some embodiments, sugar-alcohols are employed and provide a stabilizing effect.

The carbohydrate may be incorporated into any commercially available biosensor, including Freestyle Libre, Freestyle Libre2, Freestyle Libre 3, Freestyle Libre for Kids, and the Freestyle Libre Pro.

The carbohydrate may be incorporated into a Freestyle Libre biosensor described in one or more of the following patents: U.S. Pat. Nos. 7,620,438; 7,826,382; 7,920,907; 8,106,780; 8,115,635; 8,223,021; 8,280,474; 8,358,210; 8,390,455; 8,409,093; 8,410,939; 8,542,122; 8,617,069; 8,688,188; 8,737,259; 8,760,297; 8,816,862; 8,915,850; 9,000,929; 9,007,781; 9,008,743; 9,042,955; 9,060,805; 9,184,875; 9,186,098; 9,186,113; 9,215,992; 9,226,714; 9,265,453; 9,271,670; 9,314,198; 9,336,423; 9,351,669; 9,402,544; 9,402,570; 9,474,475; 9,532,737; 9,549,694; 9,636,068; 9,687,183; 9,693,713; 9,750,444; 9,808,186; 9,831,985; 9,895,091; 9,907,470; 9,931,066; 9,980,669; 9,993,188; 10,010,280; 10,028,680; 10,136,816; 10,136,845; 10,178,954; 10,201,301; 10,213,139; 10,349,877; 10,492,685; 10,653,344; 10,736,547; 10,765,351; 10,820,842; 10,923,218; 10,952,611; 10,976,304; 11,017,890; 11,051,724; 11,103,165; 11,119,090; 11,179,068; 11,202,591; 11,207,006; 11,213,229; 11,266,335; 11,272,867; 11,363,975; 11,627,898; 11,696,684; D882,432S; D903,877S; D915,601S; D915,602S; D955,432S; or RE 47,315.

The carbohydrate may be incorporated into a Freestyle Libre 2 biosensor described in one or more of the following patents: U.S. Pat. Nos. 7,620,438; 7,826,382; 7,920,907; 8,106,780; 8,115,635; 8,223,021; 8,280,474; 8,358,210; 8,390,455; 8,409,093; 8,410,939; 8,542,122; 8,617,069; 8,688,188; 8,737,259; 8,760,297; 8,816,862; 8,915,850; 9,000,929; 9,007,781; 9,008,743; 9,042,955; 9,060,805; 9,184,875; 9,186,098; 9,186,113; 9,215,992; 9,226,714; 9,265,453; 9,271,670; 9,314,198; 9,336,423; 9,351,669; 9,402,544; 9,402,570; 9,474,475; 9,532,737; 9,549,694; 9,636,068; 9,687,183; 9,693,713; 9,750,444; 9,808,186; 9,831,985; 9,895,091; 9,907,470; 9,931,066; 9,980,669; 9,993,188; 10,010,280; 10,028,680; 10,136,816; 10,136,845; 10,178,954; 10,201,301; 10,213,139; 10,349,877; 10,492,685; 10,653,317; 10,653,344; 10,736,547; 10,765,351; 10,820,842; 10,918,342; 10,923,218; 10,952,611; 10,976,304; 11,006,870; 11,006,871; 11,017,890; 11,051,724; 11,103,165; 11,119,090; 11,179,068; 11,202,591; 11,205,511; 11,207,006; 11,213,229; 11,266,335; 11,272,867; 11,298,058; 11,363,975; 11,369,740; 11,534,089; 11,627,898; D882,432S; D903,877S; D915,601S; D915,602S; D954,740S; D955,432S; D955,435S; RE47,315.

The carbohydrate may be incorporated into a Freestyle Libre 3 biosensor described in one or more of the following patents: U.S. Pat. Nos. 7,826,382; 7,920,907; 8,280,474; 8,409,093; 8,617,069; 8,688,188; 8,737,259; 8,816,862; 8,915,850; 9,000,929; 9,007,781; 9,008,743; 9,042,955; 9,184,875; 9,186,098; 9,186,113; 9,215,992; 9,226,714; 9,265,453; 9,271,670; 9,314,198; 9,351,669; 9,402,544; 9,474,475; 9,549,694; 9,636,068; 9,687,183; 9,808,186; 9,831,985; 9,895,091; 10,136,816; 10,136,845; 10,178,954; 10,201,301; 10,349,877; 10,653,317; 10,653,344; 10,786,190; 10,820,842; 10,827,954; 10,918,342; 10,923,218; 10,952,611; 10,952,653; 11,006,870; 11,017,890; 11,071,478; 11,103,165; 11,116,431; 11,152,112; 11,202,591; 11,205,511; 11,207,006; 11,213,229; 11,266,335; 11,272,867; 11,272,890; 11,298,056; 11,298,058; 11,363,975; 11,369,740; 11,534,089; 11,678,848; D882,432S; D955,433S; D958,156S; D966,333S; D967,128S; D967,136S; D982,762S; RE47,315.

The carbohydrate may be incorporated into a Libre Pro biosensor described in one or more of the following patents: U.S. Pat. Nos. 7,620,438; 7,826,382; 7,920,907; 8,106,780; 8,115,635; 8,223,021; 8,280,474; 8,358,210; 8,390,455; 8,410,939; 8,542,122; 8,617,069; 8,737,259; 8,760,297; 9,000,929; 9,007,781; 9,008,743; 9,042,955; 9,060,805; 9,184,875; 9,186,098; 9,215,992; 9,265,453; 9,271,670; 9,314,198; 9,336,423; 9,351,669; 9,402,544; 9,402,570; 9,474,475; 9,636,068; 9,687,183; 9,693,713; 9,750,444; 9,808,186; 9,831,985; 9,895,091; 9,907,470; 9,931,066; 9,980,669; 9,993,188; 10,010,280; 10,028,680; 10,136,816; 10,178,954; 10,213,139; 10,349,877; 10,492,685; 10,736,547; 10,765,351; 10,952,611; 10,976,304; 11,051,724; 11,103,165; 11,119,090; 11,179,068; 11,202,591; 11,207,006; 11,213,229; 11,264,133; 11,266,335; 11,272,867; 11,276,492; 11,363,975; D882,432S; D903,877S; D915,601S; D915,602S.

### 3. Methods of Manufacture

In some aspects, provided herein are methods of manufacturing an analyte sensor with improved stability and performance provided herein. In some embodiments, provided herein are methods of manufacturing an analyte sensor provided herein, comprising contacting the sensing layer of the analyte sensor with a carbohydrate. In some embodiments, the sensing layer comprises glucose oxidase enzyme. The sensing layer may be contacted with the carbohydrate directly (e.g. by directly contacting the sensing layer with the carbohydrate) or indirectly (e.g. by contacting the assembled sensor comprising the sensing layer and the polymeric membrane with the carbohydrate, and allowing the carbohydrate to diffuse through the polymeric membrane and into the sensing layer).

In some embodiments, the sensing layer is contacted with the carbohydrate directly. For example, in some embodiments contacting the sensing layer with the carbohydrate comprises contacting the sensing layer with a solution comprising the carbohydrate, such that the carbohydrate diffuses from the solution onto the sensing layer. In some embodiments, the sensing layer (e.g. the sensing layer containing the carbohydrate) is subsequently coated with a membrane polymer. For example, in some embodiments the sensing layer (e.g. the layer comprising the redox polymer and glucose oxidase enzyme) is dipped in a solution comprising the carbohydrate, and the carbohydrate diffuses from the solution into the sensing layer. In some embodiments, the sensing layer is contacted with the carbohydrate prior to dispensing the sensing layer onto the sensor electrode(s). In some embodiments, the sensing layer is contacted with the carbohydrate after dispensing the sensing layer onto the sensor electrode(s). In some embodiments, the sensing layer containing the carbohydrate is then dipped in a membrane solution (e.g. a solution comprising the membrane polymer and a crosslinker) to form the polymeric membrane, which coats the sensor. Exemplary membrane polymers and crosslinkers are described above. For example, in some embodiments the membrane polymer comprises a poly(vinylpyridine-co-styrene) copolymer and the crosslinker comprises an epoxide (e.g. PEGDGE, Gly3). In some embodiments, the carbohydrate is of a relatively high molecular weight (e.g. a disaccharide, an oligosaccharide, a polysaccharide), which may prevent carbohydrate loss during the manufacturing process. For example, in some embodiments the carbohydrate is of a relatively high molecular weight (e.g. a disaccharide, an oligosaccharide, a polysaccharide) and is contacted with the sensing layer prior to dipping the sensing layer in the membrane solution (e.g. prior to coating the sensing layer with the polymeric membrane), which helps to “lock” the carbohydrate into the sensing layer and prevent loss of the carbohydrate from the sensing layer during subsequent dipping of the carbohydrate-containing sensing layer into the membrane solution.

In some embodiments, the carbohydrate is water-soluble. In some embodiments, a water soluble carbohydrate is contained within an aqueous sensing layer, and the aqueous sensing layer containing the water-soluble carbohydrate is dipped into a membrane solution comprising solution in which the carbohydrate is not soluble. For example, in some embodiments the membrane solution is oil-based or alcohol-based, and the carbohydrate is not soluble in such a solution. In such embodiments, the water-soluble carbohydrate remains within the sensing layer during the dipping process of the sensing layer into the membrane solution, avoiding unwanted loss of the carbohydrate during manufacture of the sensor.

In some embodiments, contacting the sensing layer with the carbohydrate comprises contacting the sensing layer with a solution comprising the carbohydrate, a membrane polymer, and a crosslinker. For example, in some embodiments the sensing layer is contacted with the carbohydrate dipping the sensing layer into a solution comprising the carbohydrate, a membrane polymer, and a crosslinker. Exemplary membrane polymers and crosslinkers are described above. For example, in some embodiments the membrane polymer comprises a poly(vinylpyridine-co-styrene) copolymer and the crosslinker comprises an epoxide (e.g. PEGDGE, Gly3). In such embodiments, the carbohydrate diffuses from the solution into the sensing layer and the sensing layer is coated with the polymeric membrane (e.g. by crosslinking of the membrane polymer) within the same dipping process. In some embodiments, the method comprises performing multiple dipping steps. For example, in some embodiments the method comprises contacting the sensing layer with a solution comprising the carbohydrate, a membrane polymer, and a crosslinker. During this initial dipping step, the carbohydrate diffuses from the solution into the sensing layer, and the initial coating of the polymeric membrane onto the sensing layer is formed. The method may then further comprise performing one or more additional dipping steps, wherein the sensing layer is dipped into a solution comprising the membrane polymer and a crosslinker but lacking the carbohydrate. These subsequent dipping steps increase the thickness of the membrane polymer coating the carbohydrate-containing sensing layer.

In some embodiments, the sensing layer is contacted with the carbohydrate indirectly. For example, in some embodiments the analyte sensor comprises a sensing layer coated with a membrane polymer, and contacting the sensing layer with the carbohydrate comprises contacting the analyte sensor with a solution comprising the carbohydrate. The carbohydrate diffuses through the membrane polymer and into the sensing layer. For example, in some embodiments the analyte sensor comprises a sensing layer comprising an enzyme for detection of an analyte (e.g. glucose oxidase) and a redox polymer, as described above, and the sensing layer is coated with a membrane polymer (e.g. a poly(vinylpyridine-co-styrene) copolymer). In some embodiments, the analyte sensor is contacted with a solution comprising the carbohydrate, and the carbohydrate diffuses from the solution through the polymeric membrane, and onto the sensing layer. In some embodiments, some carbohydrate remains in the polymeric membrane and some carbohydrate passes through the polymeric membrane and onto the sensing layer.

For any of the methods of manufacture described herein, any suitable carbohydrate may be used. In some embodiments, the carbohydrate is a pyranose, furanose, disaccharide, oligosaccharide, polysaccharide, iminosugar, sugar alcohol, glycoside, amino sugar, or a combination thereof the term “pyranose” refers to a monosaccharide that forms a six-membered ring. In some embodiments, the pyranose is glucose, galactose, mannose, or a combination thereof. The term “furanose” refers to a monosaccharide that forms a five-membered ring. In some embodiments, the furanose is fructose, xylose, ribose, or a combination thereof. The term “disaccharide” refers to a combination of 2 pyranose and/or furanose units (e.g. two pyranose units, two furanose units, or one pyranose and one furanose unit). In some embodiments, the disaccharide is lactose, maltose, cellobiose, or a combination thereof. The term “oligosaccharide” typically refers to a saccharide containing 3-10 monosaccharide units, whereas the term “polysaccharide” typically refers to a saccharide containing more than 10 monosaccharide units. In some embodiments, the oligo- or polysaccharide is starch, maltotriose, amylose, cellulose, inulin, or a combination thereof. In some embodiments, the oxygen present in a 5 or 6 membered ring can be substituted with a suitable moiety such as carbon, nitrogen, or sulfur. For example, the carbohydrate may be the iminosugar nojirimycin. In some embodiments, the carbohydrate comprises a 5 or 6 membered ring with 3 or more hydroxyl groups and any number of additional carbon atoms attached to the ring. For example, the carbohydrate may be the sugar alcohol inositol.

In some embodiments, the iminosugar is nojirimycin. In some embodiments, the sugar alcohol is inositol. In some embodiments, the glycoside is a glucoside. In some embodiments, the glucoside is 1-O-methyl glucose. In some embodiments, the glycoside comprises an aryl group. In some embodiments, the glycoside comprises an acyl group. In some embodiments, the amino sugar is a glucosamine or N-acetylglucosamine.

In some embodiments, the carbohydrate comprises hydroxyls substituted to form O—R groups, with R including alkyl, aryl, acyl. Exemplary substituted carbohydrates include, for example, 1-O-methyl glucose, pentaacetylglucose, etc. In some embodiments, the carbohydrate comprises an exo-ring oxygen that is substituted with nitrogen, as in glucosamine or N-acetylglucosamine.

### 4. Clinical Use of Biosensors

Provided herein are biosensors with sufficient sensitivity and stability to be used as very small, subcutaneous biosensors for the measurement of clinically relevant compounds such as glucose and lactate. The biosensors provided herein comprising a carbohydrate in the sensing layer and/or the polymeric membrane are demonstrated herein to have improved performance and stability compared to equivalent biosensors lacking the carbohydrate. Accordingly, the biosensors provided herein can be used subcutaneously for a longer period of time without the need to replace the biosensor, which provides improved patient care and comfort. The electrodes accurately measure glucose in the range of about 2-30 M and lactate in the range of about 0.5-10 mM. One function of the implanted biosensor is to sound an alarm when, for example, a patient's glucose concentration is too low or too high. When pairs of implanted electrodes are used, there are three situations in which an alarm is triggered: low glucose concentration, high glucose concentration; sensor malfunction as determined by a discrepancy between paired readings of the two sensors. A discrepancy sufficient to trigger the alarm may be, for example more than two or three times the standard deviation persisting for a defined period, e.g. not less than ten minutes. Such a system may be useful in sleeping patients, and also in emergency and intensive care hospital rooms, where vital functions are continuously monitored.

Another function of the biosensors described herein is to assist diabetics in maintaining their blood glucose levels near normal. Many diabetics now maintain higher than normal blood glucose levels because of danger of coma and death in severe hypoglycemia. However, maintaining blood glucose levels substantially, e.g. approximately 40% or more above normal leads to retinopathy and blindness as well as to kidney failure. Use of the subcutaneous biosensors to frequently, if not continuously, monitor glucose concentrations is desirable so that glucose concentrations can be maintained closer to an optimum level. The subcutaneous biosensors can be used to measure the rate of rise and decline of glucose concentrations after a meal or the administration of glucose (e.g. a glucose tolerance test). The sensors are also useful in feedback loops for automatic or manually controlled maintenance of glucose concentrations within a defined range. For example, when used in conjunction with an insulin pump, a specified amount of insulin is delivered from the pump if the sensor glucose reading is above a set value.

In all of these applications, the ability to promptly confirm that the implanted sensor reading is accurate is essential. Prompt confirmation and rapid recalibration are possible only when one-point calibration is valid. Generally, even if a sensor's response is linear through the relevant concentration range, calibration requires at least two blood or fluid samples, withdrawn from the patient at times when the glucose concentration differs. It usually takes several hours for the glucose concentration to change sufficiently to validate proper functioning by two-point calibration. The ability to confirm and recalibrate using only one point is thus a highly desirable feature of the present invention.

It is preferred that the biosensors be implanted in subcutaneous tissue so as to make the sensor relatively unobtrusive, and at a site where they would not be easily dislodged, e.g. with turning or movement. It is also preferred, when readings are not corrected for temperature (which they generally are) that the sensors be implanted where they are likely to be at body temperature, e.g. near 37° C., and preferably covered by clothing. Convenient sites include the abdomen, inner thigh and arm.

Although continuous current measurement for assaying glucose is described herein, the electrical measurement by which the glucose concentration is monitored can be continuous or pulsed. It can be a current measurement, a potential measurement or a measurement of charge. It can be a steady state measurement, where a current or potential that does not substantially change during the measurement is monitored, or it can be a dynamic measurement, e.g. one in which the rate of current or potential change in a given time period is monitored. These measurements require at least one electrode in addition to the sensing electrode. This second electrode can be placed on the skin or can be implanted, e.g. subcutaneously. When a current is measured it is useful to have a potentiostat in the circuit connecting the implanted sensing electrode and the second electrode, that can be a reference electrode, such as an Ag/AgCl electrode. When a current is measured the reference electrode may serve also as the counter electrode. The counter electrode can also be a separate, third electrode, such as a platinum, carbon, palladium or gold electrode.

In addition to implanting the sensing electrode in the body, fluid from the body, particularly fluid from the subcutaneous region, can be routed to an external sensor. It is preferred in this case to implant in the subcutaneous region a microfiltration giver and pull fluid to an evacuated container, the fluid traversing a cell containing the sensing electrode. Preferably this cell also contains a second electrode, e.g. a reference electrode which may serve also as a counter electrode. Alternatively, the reference and counter electrodes may be separate electrodes. In coulometric measurements only two electrodes, the sensing electrode and the counter electrode are required. The flow of body fluid may be pulsed or continuous. Other than an implanted microfiltration fiber, also a microdialysis fiber may be used, preferably in conjunction with a pump.

The present invention is applicable to corded or cabled glucose-sensing systems, as described above, as well as other analyte-sensing or glucose-sensing systems. For example, it is contemplated that suitable results, along the lines of those described herein, may be obtained using a wireless glucose-sensing system that comprises a pager-sized, hand-held, informational display module. For example, a FREESTYLE NAVIGATOR wireless glucose-sensing system or a Freestyle Libre glucose-sensing system is applicable to the sensors described herein. The glucose-sensing system may be capable of providing real-time glucose information at 1-minute intervals and information regarding rates and trends associated with changes in glucose levels. This system is further capable of providing a visual indication of glucose level rates, allowing users to discriminate among glucose rate changes of less than 1 mg/dL per minute, 1-2 mg/dL per minute (moderate change), and greater than 2 mg/dL per minute (rapid change). It is contemplated that sensors having features such as these will be advantageous in bringing information of predictive or diagnostic utility to users. The system may also be designed to provide hypoglycemic and hyperglycemic alarms with user-settable thresholds.

### Sensor Configuration

The subcutaneous portion of the sensor may be placed into the subcutaneous tissue of the upper arm or the abdomen of a subject or patient using a spring-actuated insertion mechanism. (See: U.S. Patent Application Ser. No. 60/424,099 of Funderburk et al. filed Nov. 5, 2002; and U.S. Patent Application Publication No. 2004/0133164 A1 of Funderburk et al. filed Nov. 5, 2003). The sensor may be connected via a cord to a portable, potentiostat-data logger device which may be used to maintain the glucose-sensing working electrode at a potential of +40 mV versus the Ag/AgCl reference electrode, while obtaining and storing instantaneous current values at 10-second intervals.

In some embodiments, a portion of the sensor is transcutaneously inserted into the subcutaneous space. For example, in some embodiments the major portion of the sensor is above the surface of the skin, with an insertion tip penetrating through the skin and into the subcutaneous space, where it is bathed in biofluid. In some embodiments, at least a portion of the working electrode, reference electrode, and counter electrode are at the end of the insertion tip, which is inserted during use. In some embodiments, at least a portion of the working electrode, reference electrode, and counter electrode remain above the skin surface during use.

In some embodiments, the insertion tip comprises the working electrode, the sensing layer, and the polymeric membrane covering the sensing layer. The tip is inserted in the subcutaneous space and bathed in the surrounding biofluid.

In some embodiments, the sensor comprises a head portion which is attached to a body portion that includes a housing that encloses other parts that contribute to the sensor function, such as a circuit board and microprocessor for processing electrochemical input into an informative signal, a battery for power, and an antenna for sending signal to an external device. Three electrodes, a working electrode, a reference electrode, and a counter electrode may be surrounded for part of their length within the ceramic head portion of the sensor, with one end of each penetrating the surface of the head portion, and the other end of each extending into the interior of the body of the sensor. In some embodiments, the most distal portion of the head portion of the sensor is the sensing layer, where the electrodes terminate. In some embodiments, the sensing layer includes glucose oxidase, which recognizes glucose and initiates the first step in transduction of the glucose concentration into an informative signal. In some embodiments, the external portion of the sensing layer is exposed to the surrounding biofluid. In some embodiments, the sensing layer is covered with a polymeric membrane which is exposed to the surrounding biofluid.

### EXAMPLES

### Example 1

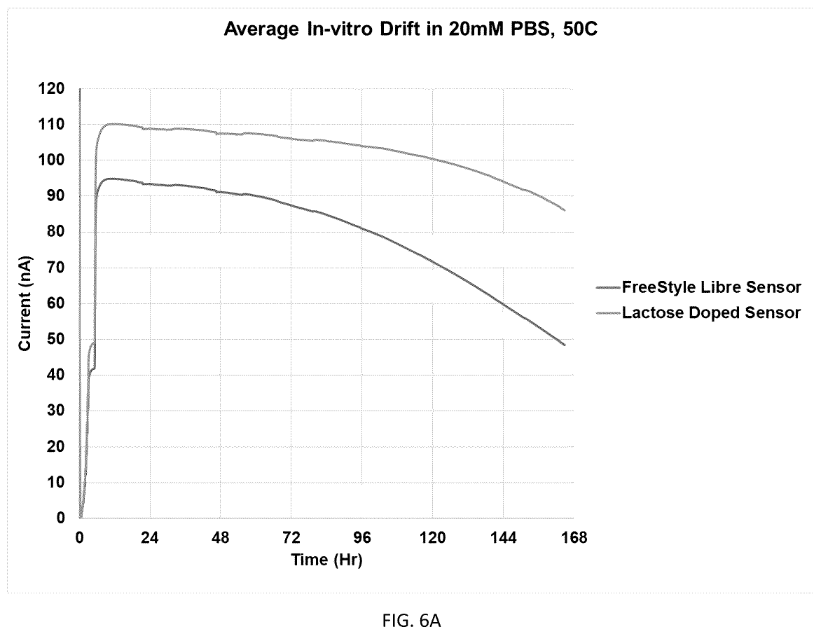

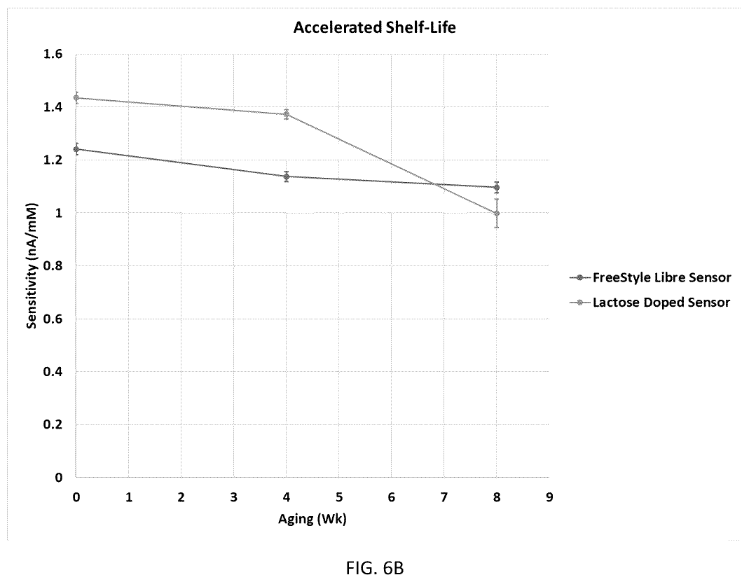

FreeStyle Libre sensors were prepared and compared to sensors with 50 mM glucose added to the membrane dipping solution. Results are shown in FIG. 6. As shown in FIG. 6A, a clear improvement in drift stability was seen in glucose doped sensors. Drift stability was tested at 37 C in 30 mM glucose solution. As shown in FIG. 6B, glucose doped sensors also had improved shelf life stability. Shelf life stability was tested at 56 C for accelerated shelf-life testing.

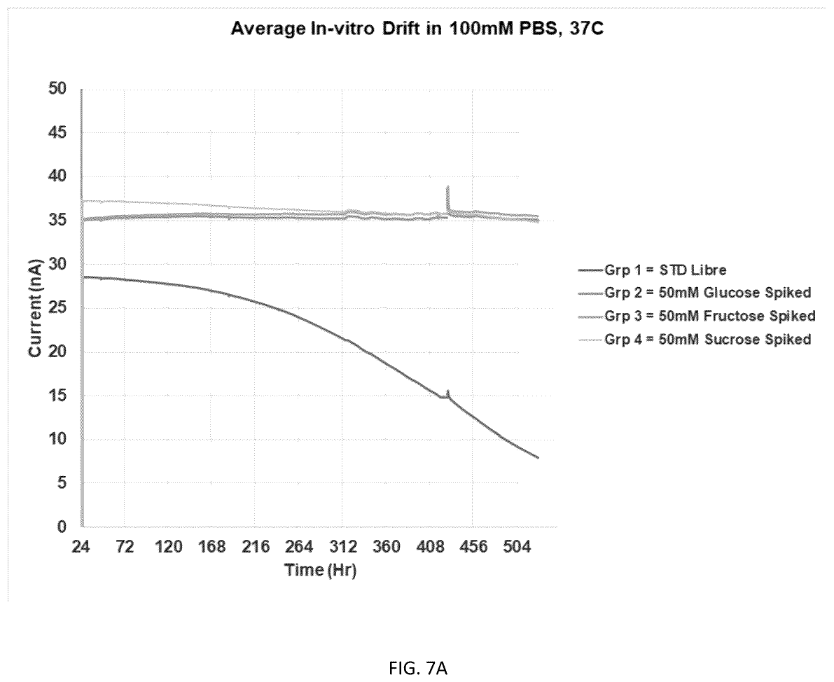

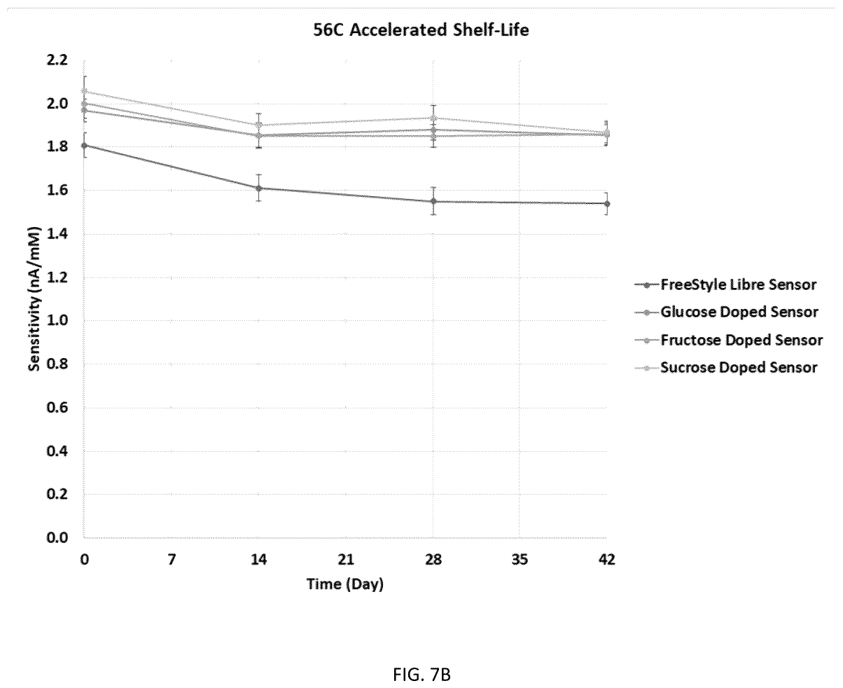

Stabilization with carbohydrates other than glucose was subsequently evaluated. FreeStyle Libre sensors were prepared and compared to sensors with 50 mM glucose, fructose or sucrose added to the membrane dipping solution. Results are shown in FIG. 7. As shown in FIG. 7A, a clear improvement in drift stability (tested at 37 C in 30 mM glucose solution) and shelf life stability (tested at 56 C—accelerated testing) for glucose as well as fructose and sucrose addition was observed.

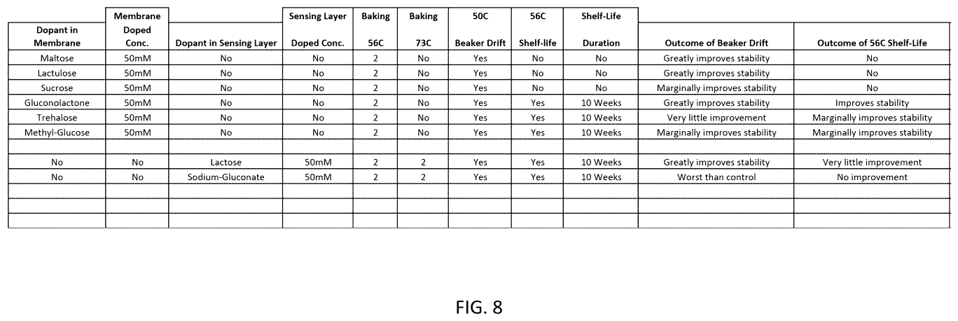

The impact of a carbohydrate dopant in the membrane polymer and/or in the sensing layer was further evaluated. The carbohydrates maltose, lactulose, sucrose, gluoconolactone, trehalose, and methyl-glucose were tested by addition to the polymeric membrane, with no additional carbohydrate specifically added to the sensing layer. However, diffusion from the polymeric membrane to the sensing layer is expected to occur. The carbohydrates lactose and sodium gluconate were tested by addition to the sensing layer, with no additional carbohydrate dopant directly added to the polymeric membrane. Drift stability and shelf-life were evaluated. Results are shown in FIG. 8.

Each of the various references, presentations, publications, provisional and/or non-provisional U.S. patent applications, U.S. patents, non-U.S. patent applications, and/or non-U.S. patents that have been identified herein, is incorporated herein in its entirety by this reference. Although various aspects and features of the present invention may have been described largely with respect to applications, or more specifically, medical applications, involving diabetic humans, it will be understood that such aspects and features also relate to any of a variety of applications involving non-diabetic humans and any and all other animals. Further, although various aspects and features of the present invention may have been described largely with respect to applications involving partially implanted sensors, such as transcutaneous or subcutaneous sensors, it will be understood that such aspects and features also relate to any of a variety of sensors that are suitable for use in connection with the body of an animal or a human, such as those suitable for use as fully implanted in the body of an animal or a human. Finally, although the various aspects and features of the present invention have been described with respect to various embodiments and specific examples herein, all of which may be made or carried out conventionally, it will be understood that the invention is entitled to protection within the full scope of the appended claims.

1. An analyte sensor comprising a sensing layer and a membrane polymer, wherein the sensing layer comprises glucose oxidase enzyme and wherein the sensing layer and/or the membrane polymer comprise a carbohydrate.

2. The analyte sensor of claim 1, wherein the sensing layer comprises a redox polymer.

3. The analyte sensor of claim 2, wherein the redox polymer comprises a transition metal complex, a polymeric backbone, and a crosslinker.

4.-17. (canceled)

18. The analyte sensor of claim 1, wherein the carbohydrate is a pyranose, furanose, disaccharide, oligosaccharide, polysaccharide, iminosugar, sugar alcohol, glycoside, amino sugar, or a combination thereof.

19. The analyte sensor of claim 18, wherein the pyranose is glucose, galactose, mannose, or a combination thereof.

20. The analyte sensor of claim 18, wherein the furanose is fructose, xylose, ribose, or a combination thereof.

21. The analyte sensor of claim 18, wherein the disaccharide is lactose, maltose, cellobiose, or a combination thereof.

22. The analyte sensor of claim 18, wherein the oligo- or polysaccharide is starch, maltotriose, amylose, cellulose, inulin, or a combination thereof.

23. The analyte sensor of claim 18, wherein the iminosugar is nojirimycin.

24. The analyte sensor of claim 18, wherein said sugar alcohol is inositol.

25. The analyte sensor of claim 18, wherein said glycoside is a glucoside.

26. The analyte sensor of claim 25, wherein the glucoside is 1-O-methyl glucose.

27. The analyte sensor of claim 18, wherein the glycoside comprises an aryl group.

28. The analyte sensor of claim 18, wherein the glycoside comprises an acyl group.

29. The analyte sensor of claim 18, wherein the amino sugar is a glucosamine or N-acetylglucosamine.

30. A method of manufacturing an analyte sensor, the method comprising contacting a sensing layer of the analyte sensor with a carbohydrate, wherein the sensing layer comprises glucose oxidase enzyme.

31. The method of claim 30, wherein said contacting comprises contacting the sensing layer with a solution comprising the carbohydrate, followed by coating the sensing layer with a membrane polymer.

32. (canceled)

33. The method of claim 30, wherein said contacting comprises contacting the sensing layer with a solution comprising the carbohydrate, a membrane polymer, and a crosslinker.

34.-35. (canceled)

36. The method of claim 30, wherein the analyte sensor comprises a sensing layer coated with a membrane polymer, and wherein said contacting comprises contacting the electrochemical sensor device with a solution comprising the carbohydrate, wherein the carbohydrate diffuses through the membrane polymer and into the sensing layer.

37.-56. (canceled)

57. A method of monitoring blood glucose levels in a subject, comprising contacting the subject with the analyte sensor of claim 1.
